(19) United States
(12) Patent Application Publication
(10) Pub. No.: US 2015/0275254 A1
(43) Pub. Date: Oct. 1, 2015

(54) ENZYME LOADING, CELLULOSE HYDROLYSIS, AND INHIBITION OF CELLOBIOHYDROLASES USING LIQUID HOT WATER PRETREATMENT

(71) Applicant: PURDUE RESEARCH FOUNDATION, West Lafayette, IN (US)

(72) Inventors: Michael Ralph Ladisch, West Lafayette, IN (US); Nathan Scott Mosier, West Lafayette, IN (US); Young Mi Kim, Woodbury, MN (US); Eduardo de Aquino Ximines, West Lafayette, IN (US); Thomas Richard Kreke, West Lafayette, IN (US); Ja Kyong Ko, West Lafayette, IN (US)

(73) Assignee: PURDUE RESEARCH FOUNDATION, West Lafayette, IN (US)

(21) Appl. No.: 14/670,564

(22) Filed: Mar. 27, 2015

Related U.S. Application Data
(60) Provisional application No. 61/971,408, filed on Mar. 27, 2014.

(51) Int. Cl. C12P 19/14; C12P 19/02

(52) U.S. Cl. CPC C12P 19/14; C12P 19/02; C12P 2201/00

(57) ABSTRACT

Disclosed herein are methods for extracting sugars from cellulose-containing sources to achieve high glucose yields while greatly reducing the amount of cellulase enzyme needed.

### CROSS-REFERENCE TO RELATED APPLICATIONS

The present U.S. patent application is related to and claims the priority benefit of U.S. Provisional Patent Application Ser. No. 61/971,408, filed Mar. 27, 2014, the contents of which is hereby incorporated by reference in its entirety into this disclosure.

### STATEMENT OF GOVERNMENT INTEREST

This invention was made with government support under DE-AC36-99G010337 DOE DE-FG02-06ER06-03, G012026-174, and DE-FG02-06ER64301 awarded by the US Department of Energy; DOE Cooperative Agreement G018103 awarded by the US Department of Energy; and USDA Hatch Project 10677 and 10646. The government has certain rights in the invention.

### TECHNICAL FIELD

This disclosure relates to improving glucose yield from biomass, and in particular to a method for decreasing the inhibitory effects of lignin and modifying lignin structure with liquid hot water pretreatment techniques.

### BACKGROUND

This section introduces aspects that may help facilitate a better understanding of the disclosure. Accordingly, these statements are to be read in this light and are not to be understood as admissions about what is or is not prior art.

Liquid hot water (LWH), steam explosion, and dilute acid pretreatments enhance enzyme hydrolysis of cellulose in lignocellulosic biomass. These pretreatments remove hemicellulose and some lignin as well as alter other biomass characteristics including particle size, porosity, pore size, swelling, fiber size, cellulose crystallinity, degree of polymerization (DP) of cellulose, accessible surface area and charge, microstructure of the cell wall and composition. Despite numerous studies, definitive correlation of properties of lignocellulosic biomass with the extent of enzyme hydrolysis, i.e., recalcitrance, has remained elusive. This is caused, in part, by use of different measures of biomass properties for different lignocellulosic feedstocks pretreated at different conditions. This has hindered comprehensive and internally consistent comparisons of enzyme hydrolysis as a function of changes in plant cell wall structures resulting from pretreatment. There is currently an unmet need for a method for decreasing the inhibitory effects of lignin and a method of modifying the lignin structure.

### SUMMARY

Disclosed herein is a method for enhancing enzyme hydrolysis. In one embodiment, the method involves pretreating a starting material at a temperature range to produce a pretreated starting material and introducing a non-catalytic protein into the pretreated starting material to thereby enhance e enzyme hydrolysis. The enzyme is a cellulase enzyme. The starting material is a cellulsose-containing material. The temperature range is about 220° C. to about 230° C. The non-catalytic protein is derived from at least one of vegetable matter, food processing waste or co-product streams, corn (gluten and zein), corn processing effluent from dry mill, wet mill, or fermentation processes. The vegetable matter comprises soybeans. The fermentation processes utilize corn as a feedstock, residual protein form microbial fermentation of sugar cane or sugar beet to ethanol.

### BRIEF DESCRIPTION OF DRAWINGS

FIG. 1A shows the correlation between glucose yield and enzyme accessible surface area as measured by cellulase protein and BSA adsorption for pretreated, hot-water washed mixed hardwood, specifically Spezyme CP adsorption at constant total enzyme loading of 20 FPU (32 mg)/g glucan (untreated and 1 to 3, R²=0.7146, y=0.0207x−0.119; 4 to 9, R²=0.4282, y=0.0558x−0.7375).

FIG. 1B shows the correlation between glucose yield and enzyme accessible surface area as measured by cellulase protein and BSA adsorption for pretreated, hot-water washed mixed hardwood, specifically the BSA adsorption at constant total BSA loading of 150 mg/g glucan (untreated and 1 to 3, R²=0.8665, y=0.028x+0.024; 4 to 9, R²=0.4058, y=0.0775x−0.3068).

FIG. 2A is a schematic representation of physical factors that determine enzyme access and hydrolysis of LHW mixed hardwood, showing the increase in number of particles and surface area per unit weight of pretreated hardwood accompanied by decrease in particle size.

FIG. 2B is a schematic representation of physical factors that determine enzyme access and hydrolysis of LHW mixed hardwood, depicting the combined effects of hemicellulose removal, change in porosity, and enhanced accessibility to enzymes.

FIGS. 3A-3F are SEM micrographs of untreated (FIGS. 3A and 3B, magnification 10k× and 20k×), liquid hot water pretreated hardwood at severity of log Rₒ=11.56 (FIGS. 3C and 3D, 10k× and 20k×), and log Rₒ=12.51 (FIGS. 3E and 3F, 10k× and 40k×).

FIG. 4A is a bar graph showing enzymatic hydrolysis of liquid hot water pretreated solids with and without BSA (Hydrolysis conditions: Pretreated solids of severity factor of log Rₒ=10.44, 11.39, 11.56 and 12.51 and Avicel).

FIG. 4B is a bar graph showing enzymatic hydrolysis of liquid hot water pretreated solids with and without BSA and Avicel in the presence of isolated lignins were pre-incubated with 50 mg BSA/g dry solid for 1 h at 25° C. and 200 rpm (after pre-incubation, Cellic Ctec2 of 5 FPU/g glucan was added to reaction mixture and further incubated for 72 hrs at 50° C. and 200 rpm).

FIG. 5 is a SDS-PAGE analysis of free proteins (Cellic Ctec2) in the supernatant after the 1.5 hr-adsorption experiment with isolated lignin of severity log Rₒ=12.51 (Mw, Molecular weight standards; Enzyme control, sample without lignin; Lignin control, sample without enzyme (liquid fraction); the band numbers with arrows indicate the proteins of the specific molecular weight).

FIG. 6 shows results of enzymatic hydrolysis of LHW pretreated hardwood (severity log Rₒ=12.51) and Avicel at different hydrolysis conditions (Control (CT): Enzymatic hydrolysis was performed with Cellic Ctec2 of 5 FPU (8 mg protein)/g glucan in 0.05 M citrate buffer (pH 4.8) for 72 hrs at 50° C. and 200 rpm. The substrate solids were hydrolyzed (point a) at pH 4.8 with 200 mM NaCl; (point b) at pH 5.5; (point c) at pH 4.8 with additional β-G (Novozyme 188) of 10 mg protein/g glucan; (point d) with β-G of 50 mg protein/g glucan; and (point e) pre-incubated with 50 mg BSA/g dry solid before hydrolysis (* denotes adaptation from previous study by Ko et al. (2014))).

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Lignocellulosic biomass including wood residues, agricultural wastes and energy crops have been used as renewable resources for producing biofuels. Among biomass, wood materials containing lignin of 15-40% are structurally stronger and more rigid than grass with a low lignin content of <15%. Lignins differ in structure depending on the plant source and are often classified according to the precursors of this polymer. Guaiacyl-syringyl (G-S) lignin, derived from coniferyl alcohol and sinapyl alcohol, is mostly found in hardwood species while softwood lignin consists predominantly of coniferyl alcohols. Since lignin is a three dimensional and highly-branched polymer, the different composition of monolignol units affects the structural linkage types and therefore, differs in its characteristics. Beyond wild type plant sources, lignin structure can be varied by developing transgenic plants and regulating its S/G ratio or decreasing the amount of it in the plant cell wall.

Many pretreatment strategies focus on lignin removal from biomass to achieve more efficient enzymatic hydrolysis process. For example, alkaline pretreatments using aqueous ammonia or sodium hydroxide enhance lignin degradation by cleavage of aryl-ether bonds. However, hydrothermal pretreatments using liquid hot water, steam explosion or dilute acid retain most of insoluble lignin in the pretreated biomass. Dilute acid pretreatment often results in the higher content of acid-insoluble lignin than that of the initial material. The net increase in lignin content has been assumed to be caused by the simultaneous depolymerization and repolymerization reactions of lignin accompanying the changes in lignin structure. The lignin properties which might be altered during pretreatment can be evaluated by measuring the glass transition temperature. Previous work in pulping and for the formulation of bio-plastics have characterized the glass transition behavior of lignin in which it transforms from a hard or glass-like state into a rubbery or viscous state upon heating.

The inhibitory role of lignin on enzymatic hydrolysis of cellulose has been recognized and its mechanism is generally accepted as follows. Lignin limits the enzyme access to cellulose by forming a physical barrier. It also non-productively adsorbs enzymes thereby reducing the amount of active enzymes. The nature of lignin varies depending on the biomass type, pretreatment or isolation method and is believed to influence the non-productive adsorption of enzymes.

To demonstrate the principles set forth in the present invention, in one embodiment, mixed hardwoods were pretreated with liquid hot water at different severities and then thoroughly washed to remove lignin derived, soluble inhibitors of enzyme hydrolysis.

In addition, liquid hot water (LHW) pretreatment improves enzymatic hydrolysis of cellulose by solubilizing xylan, increasing porosity, and decreasing particle size. These structural changes of lignocellulose during the LHW pretreatment lead to enhanced enzymatic accessibility of cellulose. However, the possibility of non-productive adsorption of cellulases onto lignin is also increased since most of lignin still remains in hydrothermally pretreated solids. Several efforts have been devoted to increase the enzymatic hydrolysis by blocking the exposed lignin surface using bovine serum albumin (BSA) and surfactants. The favorable effect of reducing non-productive adsorption of cellulases to lignin using lignin blocking agents at high enzyme loadings (10-20 FPU) was observed for the enzymatic hydrolysis of steam pretreated softwoods.

An alternative embodiment has been applied to reduce the recalcitrance of lignin by genetically modifying its structure as well as reducing its content in plants. Among the various lignocellulosic feedstocks, softwood lignin which is almost exclusively composed of guaiacyl (G) unit is known to be more inhibitory to enzymatic hydrolysis than hardwood and grass lignins which have a lower guaiacyl content. Hydrothermal pretreatment also alters lignin's structure into more condensed and syringyl-deficient forms. Since the structure of lignin affects the interactions with cellulases, the modification of lignin biosynthetic pathway can be a promising strategy to reduce the recalcitrance of cellulose to enzymatic hydrolysis.

Study of the underlying inhibitory mechanism of lignin was motivated by reports of non-productive cellulase adsorption onto lignins. To examine the enzyme-lignin interactions, lignins were isolated from LHW pretreated hardwoods of four different pretreatment severities. The Langmuir adsorption constants were obtained and compared to assess how the changes in lignin structure due to pretreatment severity affected the enzyme adsorption. Since enzyme components have different modes of action on cellulose, the specificity of enzyme adsorption was also examined. The enzymatic hydrolysis of LHW pretreated hardwood was evaluated based on the results from enzyme-lignin interactions.

Changes were found in recalcitrance as a function of lignocellulose properties for mixed hardwoods pretreated using liquid hot water at 4 different severities and 7 different temperatures. Recalcitrance was measured based on extents of cellulose hydrolysis using excess cellulase enzymes at a loading of 20 FPU/g glucan to carry out hydrolysis for either 72 or 168 hours. Cellulose conversions, ranging from 8 to 90%, were compared to particle size, the amount of xylan removed, porosity, pore size, degree of polymerization, lignin content, and protein adsorption. The resulting correlations were used to define parameters that determine recalcitrance, and to devise and demonstrate an approach that decreased the amount of enzyme required for hydrolysis by a factor of 10.

A total of 9 different liquid hot water (LHW) pretreatment conditions at 4 severities resulted in glucose yields of 20 to 90% compared to a hydrolysis yield for untreated hardwood of 8% (Table 1 and Table 2). Glucose yields from enzyme hydrolysis were used as the metric for analyzing impact of changes in substrate characteristics on recalcitrance.

TABLE 1
Compositions of mixed hardwood solids.

| | | After Pretreatment, Before Hydrolysis¹ | | | | | | | | |
|---|---|---|---|---|---|---|---|---|---|---|
| % | Initial | Single Stage | | | | Two Stage | | Three Stage | | |
| Component | UT | 1 | 2 | 3 | 4 | 5 | 6 | 7 | 8 | 9 |
| Glucan | 39.8 | 54.1 | 55.7 | 54.8 | 62.0 | 60.6 | 62.7 | 64.5 | 64.9 | 69.9 |
| Xylan | 16.6 | 8.2 | 6.6 | 0.0 | 0.0 | 0.0 | 0.0 | 0.0 | 0.0 | 0.0 |
| Acetyl | 2.7 | 0.0 | 0.0 | 0.0 | 0.0 | 0.0 | 0.0 | 0.0 | 0.0 | 0.0 |
| Ash | 2.0 | 0.0 | 0.0 | 0.0 | 0.0 | 0.0 | 0.5 | 0.0 | 0.1 | 0.1 |
| Lignin | 31.0 | 36.8 | 37.0 | 37.5 | 37.4 | 35.0 | 36.8 | 33.1 | 34.0 | 33.8 |
| Mass Closure | 92.1 | 99.2 | 99.3 | 92.3 | 99.4 | 95.6 | 100.1 | 97.6 | 99.1 | 103.8 |

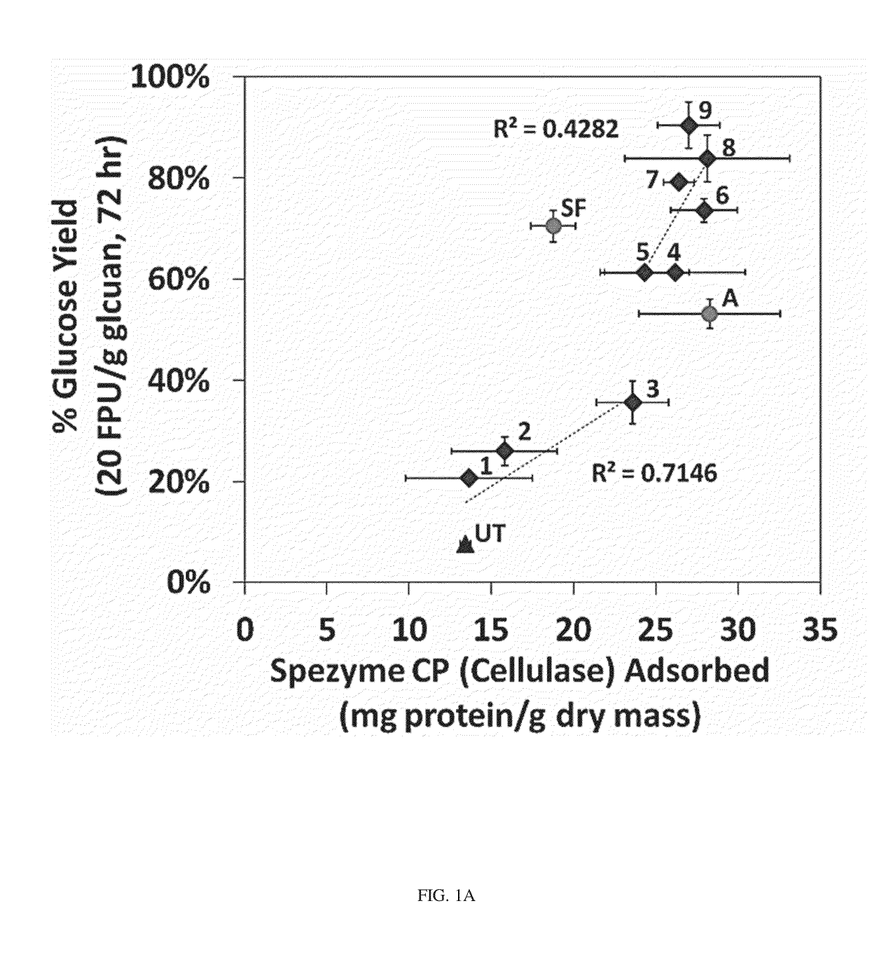

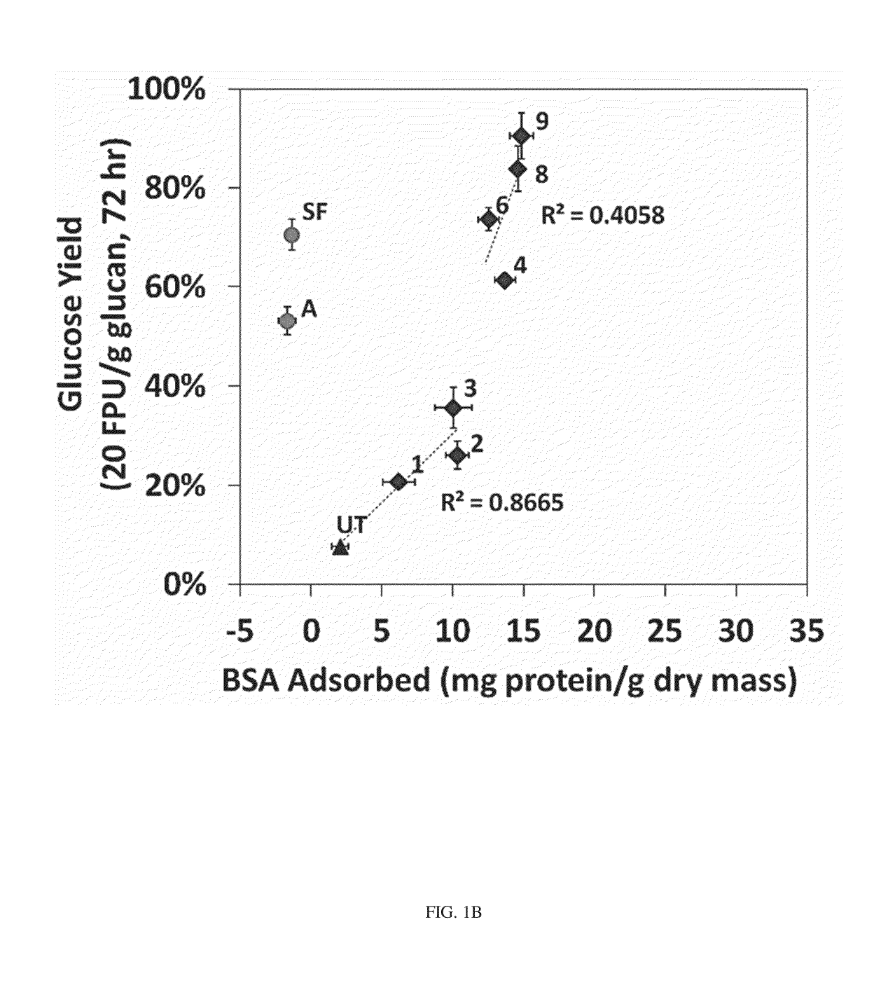

¹Columns denoted UT and 1 through 9 correspond to data points plotted in FIGS. 1A-1B.

TABLE 2
Pretreatment Conditions for Mixed Hardwood Pin Chips.

| | | Pretreatment | | | | | | | | |
|---|---|---|---|---|---|---|---|---|---|---|
| | Data | Stage 1 | | Stage 2 | | Stage 3 | | Severity Factor | | |
| Type of | Point | Temp | Time | Temp | Time | Temp | Time | OCG¹ | Kim² | Glucose³ |
| Pretreatment | ♦ | C. | min | C. | min | C. | min | (7) | (6) | Yield % |
| Single Stage | 1 | 140 | 1140 | — | — | — | — | 4.24 | 6.79 | 21 |
| | 2 | 160 | 300 | — | — | — | — | 4.24 | 8.08 | 26 |
| | 3 | 180 | 78 | — | — | — | — | 4.24 | 9.37 | 36 |
| | 4 | 200 | 20 | — | — | — | — | 4.24 | 10.64 | 61 |
| Two Stage | 5 | 180 | 20 | 210 | 5 | — | — | 4.12 | 10.98 | 61 |
| | 6 | 180 | 20 | 220 | 10 | — | — | 4.59 | 12.21 | 74 |
| Three Stage | 7 | 180 | 20 | 210 | 5 | 210 | 30 | 4.81 | 11.82 | 70 |
| | 8 | 180 | 20 | 210 | 5 | 220 | 15 | 4.81 | 12.41 | 84 |
| | 9 | 180 | 20 | 210 | 5 | 230 | 7.5 | 4.81 | 13.03 | 90 |

Untreated Pin Chips (▴, UT); Avicel PH101 (, A); Solka Floc (, SF).
¹Severity calculated from Overend, Chornet, Gascoigne (7);
²Severity from Kim et al. (6) with ω = 4.6;
³Hydrolysis at 20 FPU cellic ctec 2/g glucan, pH 4.8 to 5.0, in 50 mM citrate buffer, 50° C., 200 rpm for 72 hours with loading equivalent to 1% w/v glucan.

Impact of Xylan Content on Enzyme Hydrolysis of LHW Pretreated Mixed Hardwood:

Liquid hot water pretreatment solubilizes and removes xylan, acetyl (acetic acid), and ash from lignocellulose (compare columns UT to 1, 2, 3 in Table 1). Single-stage pretreatments at 140 and 160° C. for 19 and 5 hr at severities of log R₀=6.79 and 8.08, respectively, partially solubilized these hemicellulose components and resulted in 21 to 26% glucose yields for the pretreated, washed solids (Table 2). A pretreatment temperature of 180° C. at severity 9.37 resulted in 36% glucose yields, while at 200° C., at severity 10.64, glucose yield increased to 61%. Pretreated, washed solids from multi-stage pretreatments contained no measurable xylan, while glucose yields varied from 61 to 90% (data points 5 to 9). The correlation of Kim et al. was used to define severity since it accurately correlates changes in cellulose reactivity over the temperature range of 140 to 230° C.

While a lower xylan content corresponds to higher cellulose hydrolysis, solubilization of all of the xylan during pretreatment does not correspond to complete nor consistent cellulose conversion. Conversely, xylan containing substrate does not necessarily give a low yield as shown by Solka Floc, a lignin-free cellulose with 20% xylan. Hemicellulose removal and increased pretreatment temperatures contribute to enhanced cellulose digestibility of liquid hot water pretreated lignocelluloses. However, changes other than xylan removal also directly affect glucose yields. These results prompted us to look for other pretreatment-induced changes in substrate characteristics that would correlate to cellulose hydrolysis yields: enzyme accessible surface area, porosity, particle size, DP, and lignin effects.

Impact of Enzyme Accessible Surface Area on Enzyme Hydrolysis LHW Pretreated Mixed Hardwood:

Cellulase-accessible surface area is cited as one of the most important substrate characteristics governing the ease of enzymatic hydrolysis of lignocellulosic feedstocks. The physically accessible volume and surface area may be measured by molecular probes of known molecular weights and sizes: water, dyes, cellulase enzymes, and bovine serum albumin (BSA).

Water Retention Value (WRV):

Water retention value (WRV) measures water binding and swelling capacity which is a function of the porosity of cellulose (30). Pretreated, washed hardwood gave 1.3 to 1.4 times higher values of WRV than untreated wood. WRV does not differentiate between large enzyme accessible pores and smaller non-accessible pores since water molecules are much smaller than cellulase enzymes (MW of >40 kD). Hence, an increase in WRV of 1.6 to 1.8, alone, does not explain the rise in glucose yields from 20 to 90%.

Simon Staining.

The limiting size of pores for effective hydrolysis of lignocellulosic materials is 4-9 nm (average. 5.1 nm) and corresponds to the size of cellulases. An increase in adsorption of orange dye from 30 to 43 mg/g total solids corresponded to an increase in pore sizes in the 5 to 36 nm range and an increase in glucose yields from 8% for untreated chips to 90% for pretreated hardwoods. The amount of orange dye adsorbed by lignin-free celluloses Avicel (A) and Solka Floc (SF), also correlated to glucose yields.

The adsorbed blue dye (BD) represents the sum of small and large pores while adsorption of orange dye corresponds to large pores potentially accessible to cellulases. An increase in the ratio of OD to BD often correlates to an increased extent of hydrolysis although this was not the case for the current work with LHW pretreated hardwood. Yields increased from 8% to 90% while the OD:BD ratio decreased from about 2.3 to 1.8. Changes in porosity measured by Simon Staining do not fully explain improvements in yield, particularly for yields above 36%.

Cellulase and BSA Adsorption on Cellulose and LHW Pretreated Mixed Hardwoods:

Adsorption of cellulase and extents of hydrolysis increase with severity of LHW pretreatment (points 1 to 9 in FIG. 1A). At 25° C. untreated hardwood pin chips adsorbed 13 mg enzyme protein/g dry mass (data point 1) while the highest severity (13.03) pretreated wood chips adsorbed twice as much at 27 mg/g dry mass (data point 9 in FIG. 1A). The surface area specific to unproductive binding of cellulase to lignin was estimated by incubating the samples with a non-specific binding protein, bovine serum albumin (BSA). BSA gave a loading of 2 mg protein/g untreated hardwood solids compared to 15 mg/g for the most severely pretreated lignocellulase (FIG. 1B) out of 150 mg BSA protein/g glucan added. BSA or peptone may moderate non-specific binding of cellulase and improve enzyme hydrolysis as reported by Pan et al. and Yang and Wyman, and discussed below.

FIGS. 1A and 1B together show that LHW pretreatments increased both cellulase and non-enzyme protein (BSA) maximum binding by about 15 mg/g dry solids, corresponding to a two-fold increase in binding of cellulase protein and a 7-fold increase in BSA binding. The difference in maximal loading of enzyme (i.e., 28 mg/g, FIG. 1A) and BSA (15 mg/g dry mass, FIG. 1B) gives a measure of enzyme that binds on surfaces of cellulose, only. The amount of BSA adsorbed represents the enzyme bound on lignin surfaces. Non-specific binding to lignin increases with pretreatment severity (FIG. 1B). Evidence for protein adsorption on lignin is also provided by a negative control, i.e., lack of binding of BSA on Solka Floc and Avicel (A and SF in FIG. 1B) compared to cellulase at 19 and 28 mg/g (A and SF in FIG. 1B). A positive indication is provided by the effect of a relatively small increment in enzyme protein loading from 24 to 27 mg/g glucan on pretreated hardwood that increased conversion by 50%.

Particle Size and Extent of Hydrolysis:

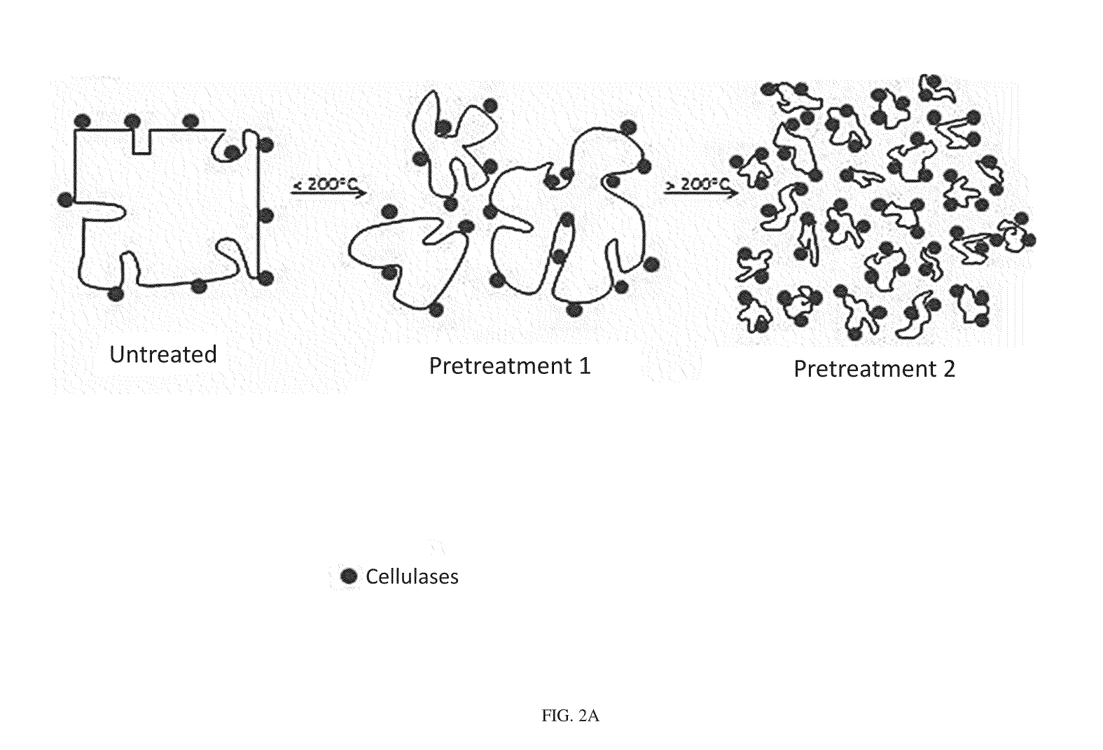

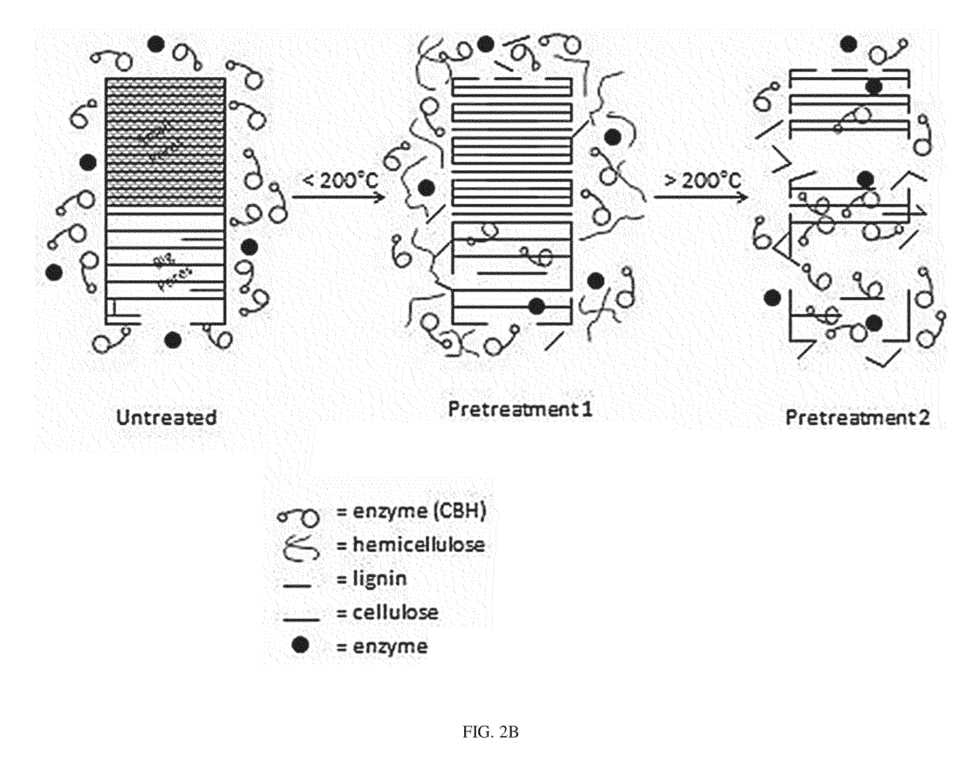

The average particle size of single stage pretreated materials was relatively constant (2.9 to 3.1 mm) while multi-stage pretreatments resulted in particles with measurably smaller sizes (2 to 2.9 mm) as well as particle size distributions characterized by a wide range of small. Glucose yield from pretreated hardwoods increased by 50% when the average wet particle size decreased from 3.0 mm to 2.0 mm and the amount of fines (less than 0.3 mm diameter) increased from 4.6% (untreated) to 14% of the total mass (pretreated solids). More than 50% of the total untreated mixed hardwood (UT) had a particle size greater than 6 mm, compared to only 4% for the pretreated solids. As average particle size decreased, glucose yield increased for pretreated solids from which all measurable amount of xylan had been removed (samples 4-9 R²=0.991). A smaller particle size increases specific surface area accessible to hydrolytic enzymes as represented in the cartoon shown in FIG. 2A. However, particle size reduction alone through mechanical grinding, absent other pretreatment, is a weak predictor of cellulose's susceptibility to enzyme hydrolysis. Removal of hemicellulose and changes in porosity as well as accessible surface area may also play a role (FIG. 2B).

Cellulose Degree of Polymerization (DP).

While reported effects of DP are unclear and contradictory, we found that DP was the only substrate characteristic that correlated with glucose yield for all pretreated samples over the entire range of pretreatment conditions tested. The viscosity average DP of untreated mixed hardwood pin chips at 2930 was in the same range as viscometric DP values of 3000 to 5000 reported for various types of wood. DP decreased as the pretreatment temperature increased at fixed severity. Point 9 had the lowest DP and highest glucose yield with a leveling off DP of between DP 200 to 500, similar to that found for sulfite wood pulps (between 200-400).

Crystallinity, which was not measured here, can also impact glucose yield. Solka Floc with a DP of 1535 was more digestible than Avicel (micro-crystalline cellulose) with a measured DP of 306. This is consistent with results of Fan and Lee who found that the hydrolysis rate for Avicel was lower than the less crystalline Solka Floc.

Pretreatment Temperature.

Increasing pretreatment temperature results in increasing glucose yields. At temperatures between 140 to 180° C. and at severities of 6.79 to 9.37, xylan dissolved, large pores accessible to cellulase enzymes increased, and particle size started to decrease. Glucose yields improved when the final temperature increased from 180 to 230° C. At 210° C. conversion, porosity, enzyme adsorption and glucose yields stayed the same as compared to single stage pretreatment at 200° C. despite the decreased severity (data points 4, 5 in FIG. 1A. When temperature was increased to 220° C. in a two-stage pretreatment, average particle size decreased further and glucose yield increased to 74%, while porosity, enzyme accessible pores, and enzyme adsorption showed a small change (FIG. 1A). Three stage pretreatment with a maximum temperature of 230° C. resulted in a glucose yield of 90% (Table 2).

Discussion of Combined Physical Effects.

External surface area readily accessible by enzymes is one of the yield-controlling factors of cellulose hydrolysis when pretreatment is severe enough to solubilize all of the hemicellulose and reduce particle size, thereby increasing surface area (FIGS. 2A and 2B). Dilute-acid pretreatment of mixed hardwoods at 220° C. resulted in particles which were much smaller, had more external surface area relative to particles from pretreatments at 180-200° C., and corresponded to a higher hydrolysis yield. Enzyme-sized pores of dilute acid pretreated mixed hardwood disappeared at the initial stage of enzymatic hydrolysis with enzyme-accessible surface in the later stages of hydrolysis corresponding to the external surfaces of the cellulose fibers.

Arantes and Saddler noted that total surface area, measured by Simon's staining, showed a positive correlation with enzyme hydrolysis for corn stover, Douglas fir, lodgepole pine, and hybrid poplar that had been pretreated by steam explosion or organosolv methods. These researchers concluded that enzymes penetrate large pores and then disaggregate and fragment the cellulose particles. The easily accessible cellulose is hydrolyzed first, leading to accumulation of the more resistant cellulose as described by Mansfield et al. and others. Enzyme hydrolysis of Avicel (a microcrystalline cellulose) also resulted in its fragmentation into smaller pieces as the hydrolysis proceeded.

Pretreatment achieves a reduction in particle size more quickly than enzyme action. A greater external surface area is expected to lead to a higher extent of cellulose hydrolysis since the effect of hindered enzyme diffusion and hydrolysis in pores is less. However, enzyme penetration into internal pores of larger particles from lignocelluloses pretreated at lower severities and temperatures is necessary since a larger fraction of cellulose surface area is inside the particles, and the particles themselves are larger. The integration of these concepts is represented in the diagram given in FIG. 2B. The diagram represents how the observed changes of hemicellulose dissolution (green or shaded areas), increase porosity at lower severities (denoted Pretreatment 1) and increase the number of particles and decrease particle size at higher severities (Pretreatment 2), enhance accessibility to hydrolytic enzymes. The question remains, however, of how enzyme loading can be reduced while still achieving high glucose yields.

Lignin.

The adsorption of enzyme on lignin and use of BSA, peptone or surfactants to interfere with enzyme adsorption was noted as early as 1980 by Saddler, Kawamoto, Dale and others. Nonetheless, lignin represents the ultimate pretreatment conundrum. Pretreatment enhances enzyme hydrolysis by disrupting lignin's close association with cellulose and compromising its protective effect while reducing particle size. At the same time pretreatment exposes lignin which adsorbs cellulase and reduces the amount of enzyme available for cellulose hydrolysis. While lignin-derived phenolic molecules that inhibit or deactivate cellulase enzymes may be substantially removed from pretreated lignocellulose by washing it with hot water, the exposed lignin remains to adsorb cellulases and prevents them from acting on their cellulose substrate.

Hydrolysis was carried out with Cellic Ctec2 at a loading of 2.2 FPU (3.5 mg)/g total solids (weight before pretreatment). Hydrolysis of untreated hardwood at pH 4.8 in 50 mM citrate buffer at 50° C. and 200 rpm gave less than 3% conversion. Hardwood, pretreated at 220° C. for 15 min (severity 12.5) gave about 30% conversion at both pH 4.8 and 5.5. Following pre-incubation with 50 mg BSA/g solids, enzyme hydrolysis at the same loading of 2.2 FPU/g solids and pH 4.8, 50° C. and 200 rpm, gave 90% conversion, thus indicating the effect of protein blocking unproductive adsorption of cellulase enzymes.

Another commercial cellulase, Spezyme CP further illustrates how the effectiveness of an even lower enzyme loading of 1.3 FPU/g pretreated solids is enhanced by BSA. In this case the decrease of enzyme specific activity from 1 to 0.02 mg cellulase/mg total protein corresponded to an increased glucose yield of 15% to 79%. Plots of glucose yields (not shown) at 2 vs. 20 FPU/g glucan for mixed hardwoods pretreated at progressively higher severities shows a yield at 20 FPU cellulase per g glucan, is linearly correlated with 2 FPU enzyme/g glucan when pre-incubated with 150 mg BSA/g glucan. At high enzyme loading, the interference of lignin was mitigated by an excess amount of cellulase protein that was available to adsorb to the lignin leaving sufficient enzyme to hydrolyze the cellulose. Pretreated solids exhibiting a low glucose yield at 20 FPU enzyme dose gave a low yield with 2 FPU enzyme thus aligning extents of conversion with effects of pretreatment. Additional evidence on the effect of lignin in adsorbing cellulases is given by positive controls, i.e., Avicel and Solka Floc—celluloses that do not contain lignin and which showed equivalent yields at both low and high enzyme loadings. For pretreated lignocellulose, reduction in the specific activity of the hydrolytic enzyme reduces the amount of enzyme required.

Cellulose Hydrolysis in Pretreated Hardwood at Low Enzyme Loading.

This counter-intuitive observation led to a systematic study in which total protein was held at 100 mg/g glucan with BSA ranging from 0 to 100 mg. A figure of merit and point of comparison for low enzyme loading is given by corn to ethanol processes, where enzyme doses are 0.1-0.2% of dry corn weight and correspond to a loading of 1 to 2 mg enzyme protein/g corn or 1.3 to 2.7 mg protein/g starch. Based on the hypothesis that a comparable cellulase (protein) dose is achievable using appropriately pretreated cellulose, an experiment was carried out with multi-stage, severity 13 pretreated hardwood. Soluble phenols that inhibit cellulases and deactivate f3-glucosidase were washed off of the pretreated solids before protein adsorption and enzyme hydrolysis was carried out. At the highest protein level of 100 mg/g glucan, >95% glucose yield is achieved at cellulase loadings of 30 FPU or 50 mg/g glucan (no exogeneous protein) and 9 FPU or 15 mg/g glucan (with 85 mg BSA added).

Pretreated sample 9 was chosen to further determine total protein loadings of BSA and cellulase required to achieve glucose yields of 80 to 90%. Total protein loading was varied from 10 to 100 mg/g glucan in a slurry of 1 w/v % pretreated hardwood which was then hydrolyzed for 168 hours. In the presence of BSA alone, the apparent yield was 2%, representing background glucose. When cellulase alone was added (corresponds to a cellulase to total protein ratio=1), yields varied between 13 to 97% for protein loadings ranging from 3 to 100 mg/g glucan, respectively. At 15 FPU (25 mg cellulase) protein/g glucan, the glucose yield was 85%, indicating sufficient total protein was present to compensate for unproductive binding of cellulase to lignin. Displacement of cellulase binding with BSA reduced cellulase loading for the same yield although a higher amount of BSA is needed to displace an equivalent amount of cellulase protein: for example, 44 mg BSA per g glucan was required to achieve 80% cellulose conversion for a cellulase loading of 6 mg cellulase/g glucan, compared to 97 mg BSA for 3 mg enzyme (1.8 FPU/g glucan or 1.3 FPU/g pretreated solids). The 3 mg Spezyme CP per g glucan loading is equivalent to 2.1 mg/g pretreated and washed solids or 1.2 mg/g hardwood before pretreatment.

When the baseline level of β-glucosidase of 1.3 mg/g pretreated solids is added to the amount of cellulase protein corresponding to 1.3 FPU/g pretreated solids (2.1 mg/g pretreated solids) the total was 3.4 mg enzyme protein/g pretreated and washed solids or about 1.9 mg/g hardwood before pretreatment. These findings will help to inform strategies that would reduce enzyme loadings below the 16 to 32 mg (10 to 20 FPU)/g glucan range typically reported in the cellulose hydrolysis literature and provide guidance and motivation for seeking other blocking agents or approaches that reduce binding of cellulases to lignin.

An internally consistent comparison based on a range of severities for liquid hot water pretreatment of hardwood has enabled definition of impacts of changes in pretreated materials on recalcitrance. The solubilization of hemicellulose opens up macropores and improves accessibility of enzymes in larger particle size, less severely pretreated celluloses. As severity and temperature of pretreatment increases above 200° C., there is a direct correlation between increased glucose yield, decreased wet particle size and decreased cellulose DP. Pretreatment also increases exposed lignin and release of soluble lignin products, thereby reducing yields since soluble lignin products inhibit and/or deactivate cellulase enzymes and/or β-glucosidase while insoluble lignin non-productively binds these enzymes. While soluble inhibitors may be removed by washing the pretreated material before enzyme hydrolysis is carried out, the negative impact of lignin in the solid material on apparent cellulase activity intensifies as the cellulase loading decreases and pretreatment severity increases. The majority of initial lignin in the pretreated solids is retained during hydrothermal pretreatments; liquid hot water, steam explosion, and dilute-acid. Strategies that mitigate the inhibitory effects of lignin are necessary to ensure robust and economic enzyme hydrolysis of cellulose.

Materials.

Hammer-milled mixed hardwood pin chips (average size of length and thickness of 0.5×0.1 cm) were provided by Mascoma Corporation (Lebanon, N.H.). Lignin-free celluloses, Avicel PH101 and Solka Floc® 300FCC were purchased from Sigma-Aldrich (St. Louis, Mo.) and International Fiber Corporation (Urbana, Ohio), respectively. Spezyme CP (50 FPU/mL, 82 mg protein/mL, specific activity: 0.61 FPU/mg protein) was provided by Genencor, a Danisco Division (Palo Alto, Calif.). Novozym 188 (780 CBU/mL, 152 mg protein/mL) was purchased from Sigma (Cat. No. C6150). Cellic™ Ctec 2 (120 FPU/mL, 190 mg protein/mL, specific activity: 0.63 FPU/mg protein) was donated by Novozyme, North America Inc. (Franklinton, N.C.). Protein concentration of enzyme was determined by total nitrogen analysis after trichloroacetic acid (TCA) precipitation as described by Hames. All other reagents and chemicals, unless otherwise noted, were purchased from Sigma-Aldrich.

Compositional Analysis.

Composition of biomass solids was determined by NREL (National Renewable Energy Laboratory) LAP analytical procedures. Analyzed components were glucan, xylan/galactan, arabinan, lignin, ash, and acetyl. The liquid fraction from pretreated lignocellulose was analyzed for soluble mono- and oligosaccharides by HPLC using LAP 014. The compositions for raw mixed hardwood and pretreated solids used in this study are summarized in Table 1. Avicel and Solka Floc were chosen as non-lignin containing cellulose controls. Avicel was 100% cellulose and Solka Floc was 80% cellulose and 20% xylan as measured by NREL's LAP compositional analysis. All measurements were made in triplicate.

Pretreatment.

Conditions and apparatus for liquid hot water (LHW) pretreatment are described elsewhere. For multi-stage pretreatments, pretreated slurry from the previous stage was filtered and washed with hot water (at 90° C.) prior to the next pretreatment. Only the solid fraction was carried over to the subsequent pretreatment. Pretreatment temperatures ranged from 140 to 230° C. and times from 5 min to 19 hours. The pressure in the sealed tube, generated by vaporization of a small fraction of the water, was high enough so that the remaining water was maintained in a liquid state. These conditions corresponded to severity factors (log R₀) of 6.79 to 13.03 (Table 1). The corresponding Overend/Chornet/Gascoigne (OCG) severity factors were 4.24 to 4.81. Kim's correlation was used throughout this paper since it best represented the temperature dependent response.

After each pretreatment, the reactor tube containing the wood chip slurry was cooled by quenching the tube in water for 10 min, opening the tube after cooling (no steam explosion effect), vacuum filtering the contents using Whatman® No 1 filter paper and recovering liquid and solid fractions for further analysis. Post-pretreatment washing consisted of mixing pretreated solids in 80-90° C. (hot) de-ionized water at 5% w/w dry solids for 10 min and then filtering through Whatman No. 1 filter paper. Pretreated hot washed solids, pretreatment liquids, and washates from each pretreatment were collected and analyzed for compositions as described in the previous section (compositional analysis). All pretreatments were carried out in triplicate.

Enzymatic Hydrolysis.

All hydrolysis runs were carried out in triplicate at 1% w/v glucan loading, pH 4.8 to 5.0 in 50 mM citrate buffer at 50° C. and 200 rpm in an incubator-shaker for 72 or 168 hours. Samples were taken at the end of hydrolysis for analysis by HPLC. Error bars represent 95% CI of a mean. All buffers contained 1 g/L sodium azide to prevent microbial growth during hydrolysis and analysis. The low solids loading was selected to minimize product inhibition of cellulases and β-glucosidase due to glucose and cellobiose accumulation resulting from enzyme hydrolysis. Together with long hydrolysis times this helped to facilitate side-by-side comparison of pretreatment effects and changes in cell wall structure, absent hydrolysis product inhibition effects. All hydrolysis samples were filtered and analyzed by HPLC.

High Enzyme Dose (20 FPU/g Glucan):

Untreated solids, pretreated solids, and lignin-free cellulose (Avicel PH101 and Solka Floc) were enzymatically hydrolyzed by 20 FPU Cellic Ctec 2 per g glucan (=32 mg protein/g glucan) for 72 hr. Upon completion of the hydrolysis, the hydrolysate was boiled for 10 min to deactivate the enzymes.

Low Enzyme Dose (2 FPU/g Glucan):

Untreated solids, pretreated solids, and lignin-free cellulose (Avicel PH 101 and Solka Floc) were hydrolyzed by 2 FPU Spezyme CP (=3 mg protein/g glucan) and 10 CBU Novozym 188 (equivalent to 2 mg protein/g glucan) for 168 hr. Enzymes were deactivated by boiling. The combination of Spezyme and Novozym was selected based on previous experience with these enzymes for wood derived aqueous streams.

Enzyme Diluted with Added BSA:

Severity 13.03 pretreated, hot-washed solids (sample 9 in Table 1) were hydrolyzed at protein loadings of 3 to 100 mg/g glucan where BSA was used to decrease the specific activity of Spezyme CP and to adjust the ratio of cellulase and total protein to biomass solids. BSA and Spezyme CP were added together to a slurry of pretreated, washed wood, and incubated at 50° C. in pH 4.8 50 mM sodium citrate buffer. Addition of enzyme coincided with the start time for hydrolysis. Novozym 188, which hydrolyzes cellobiose to glucose, was added to give 10 CBU or 2 mg protein per g glucan in all experiments that used Spezyme CP. The presence of a baseline amount of β-glucosidase ensured that cellobiose released by cellulose hydrolysis would be completely converted to glucose thereby resulting in an internally consistent measure of glucose yield.

Enzymatic Hydrolysis Time Course:

Untreated and pretreated solids of severity 12.51 were hydrolyzed by 5 FPU Cellic Ctec2 (=8 mg protein/g glucan) with or without BSA for 168 hr. The pretreated solid was pre-incubated with 85 mg BSA protein/g glucan (=50 mg/g dry mass) for 60 min at 25° C. before enzyme was added and hydrolysis occurred in pH 4.8 or 5.5, 50 mM citrate buffer at 50° C.

Cellulase and BSA Adsorption at Constant Protein.

Commercial cellulase (Spezyme CP) or BSA was added to 2.5 g/L glucan in untreated or pretreated lignocellulose, or lignin-free cellulose in pH 4.8, 50 mM sodium citrate buffer. BSA protein concentrations were 150 mg protein/g glucan. The mixture was incubated for 1 hr at 25° C. at 100 rpm in a reciprocating shaking incubator and then centrifuged at 10,000 rpm for 5 min. Protein concentration in the supernatant was measured using a Pierce BCA Protein Assay kit (Thermo Scientific, Rockford, Ill., Product No. 23225) and UV spectrophotometer at 562 nm. Enzyme and BSA blanks were solids-free while substrate blanks consisted of solids in buffer. The amount of protein adsorbed varied from 0 to 15 mg/g dry mass for BSA and 13 to 28 mg/g dry mass for cellulase. Protein loading was calculated from the difference between the added amount of protein and the protein remaining in the supernatant after incubation, corrected for spectral absorbance of substrate blanks. All measurements were made in triplicate.

Simon's Stain and Cellulose Porosity Measurements.

Simon's staining of lignocellulose for determining porosity was performed as described by Chandra et al. and others who have shown that extent of hydrolysis correlates with surface area. This method measures porosity of various lignocellulosic feedstocks based on the total and competitive adsorptions of two dyes of different sizes and affinities for cellulose. High molecular weight orange dye (OD) has a higher affinity for cellulose than blue dye (BD) but only penetrates large pores (5-36 nm). Blue dye has a diameter of 1 nm and accesses small pores from which orange dye is excluded. Therefore, orange dye populates large pores of a cellulosic material by displacing blue dye which has lower affinity for cellulose. The ratio of adsorbed orange dye to blue dye (i.e., OD:BD) corresponds to the ratio of large to small pores. Simon's staining is a useful surrogate for gauging enzyme accessibility for various pretreated materials but gives only a preliminary indication of pores accessible to enzymes. Enzyme uptake or adsorption by the solid substrate is the best metric.

Average Degree of Polymerization (DP) of Cellulose.

Cellulose DP was calculated from the intrinsic viscosity and composition of holocellulose using equation (1).

$$D P = {( \frac{1.65 \cdot [ η ] - 116 \cdot H}{G} )}^{1.11} \qquad ( 1 )$$

where [η] is the intrinsic viscosity (cm³/g) of holocellulose and H and G are the mass fractions of hemicellulose and glucan in the lignin containing sample, respectively. The holocellulose was prepared and its viscosity measured in triplicate as described in Hubbell and Ragauskas, ASTM, and Heiningen with compositional analysis carried out as described earlier in this section.

Wet Particle Size Determination.

Average particle size of wet pretreated and untreated pin chips was determined by wet sieving through metal wire sieves ranging from mesh size 2 (opening size 6 mm) to size 88 (0.04 mm). The samples were kept wet in water to prevent them from drying and shrinking. All measurements were done in duplicate.

Water Retention Value.

Water retention value (WRV) is a measure of water binding and swelling capacity based on water molecules that probe and indirectly determine the porosity of a tested material. Procedures were carried out based on TAPPI Useful Method UM 256 (30).

HPLC.

Hydrolysis samples were analyzed by Bio-Rad Aminex HPX-87H ion exchange column (300 mm×7.8 mm, Bio-Rad Laboratories Inc., Hercules, Calif.).

The effect of liquid hot water pretreatment severity on properties of hardwood lignin and enzymatic hydrolysis of cellulose is disclosed below.

Materials and Methods:

Mixed hardwood chips were used as the starting materials. These materials were hammer-milled and their average size was 1.0 and 0.2 cm in length and width, respectively. Raw mixed hardwood was composed of 43.2% glucan, 16.6% xylan, 31.8% lignin, 2.7% acetyl groups, 2.0% ash and 2.1% extractives. The composition of raw and pretreated solids was analyzed following NREL LAP standard procedures (Sluiter et al., 2008). Microcrystalline cellulose, Avicel PH101, was purchased from Sigma-Aldrich (St. Louis, Mo.).

Liquid Hot Water (LHW) Pretreatment:

For LHW pretreatment, stainless steel 316 tubes were filled with 9.4 g of hardwood chips (moisture content=46% (w/w)) and 24.4 ml of distilled water by achieving 15% (w/w) dry solids slurry (Kim et al., 2009). After heat-up time for 5-6 min, the tube was held for 5-15 min at 180-210° C. in a Tecam®SBL-1 fluidized sand bath. The pretreated tubes were immediately placed in warm water for 5 min for mild quenching. The liquid fraction was separated by vacuum filtration using Whatman #1 filter paper. The filtered solid was washed with 100 ml of hot DI water. The combined effects of pretreatment temperature (T) and time (t) were investigated based on the severity factor equation, log Rₒ (Rₒ=t×exp ((T−100)/ω)), but with a value of ω=4.6 instead of 14.75 found by Kim et al. to give a better correlation between severity factor and pretreatment responses. The carbohydrate composition of pretreated solids of different severities is summarized in Table 3.

TABLE 3
Effect of liquid hot water pretreatment on the composition of solid fraction.

| | | | | | | | Solid | Xylan |
|---|---|---|---|---|---|---|---|---|
| Sample | Severity | Severity | Temp. | Time | Glucan | Xylan | recovery | recovery |
| no. | factor^{a} | factor^{b} | (° C.) | (min) | (%) | (%) | (%) | (%) |
| Untreated | | | | | 39.8 | 16.6 | | |
| 1 | 8.25 | 3.05 | 180 | 5 | 40.4 | 14.0 | 93.9 | 78.9 |
| 2 | 8.55 | 3.36 | 180 | 10 | 44.2 | 13.2 | 89.9 | 71.2 |
| 3 | 8.73 | 3.53 | 180 | 15 | 47.6 | 10.7 | 80.7 | 52.2 |
| 4 | 9.20 | 3.35 | 190 | 5 | 40.4 | 14.0 | 85.5 | 71.9 |
| 5 | 9.50 | 3.65 | 190 | 10 | 49.7 | 10.2 | 79.7 | 48.8 |
| 6 | 9.67 | 3.83 | 190 | 15 | 52.8 | 9.2 | 76.5 | 42.5 |
| 7 | 10.14 | 3.64 | 200 | 5 | 46.3 | 11.8 | 74.0 | 52.7 |
| 8 | 10.44 | 3.94 | 200 | 10 | 53.5 | 8.4 | 73.7 | 37.4 |
| 9 | 10.62 | 4.12 | 200 | 15 | 56.0 | 6.3 | 69.6 | 26.3 |
| 10 | 11.08 | 3.94 | 210 | 5 | 54.0 | 7.4 | 71.6 | 32.0 |
| 11 | 11.39 | 4.24 | 210 | 10 | 55.9 | 6.3 | 71.1 | 27.0 |
| 12 | 11.56 | 4.41 | 210 | 15 | 57.5 | 4.0 | 72.7 | 17.5 |

^{a}Severity factor with ω = 4.6 (Kim et al., 2013)
^{b}Conventional severity factor with ω = 14.75 (Overend and Chornet, 1987)

Enzymatic Hydrolysis:

Cellic Ctec 2 with a protein content of 190 mg/mL was provided by Novozyme, North America Inc. (Franklinton, N.C.) and its activity was determined to be 118 FPU/mL. The enzyme activities were measured using IUPAC method (Ghose, 1987) and the released glucose amount was quantified by HPLC using an ion exchange Bio-Rad Aminex HPX-87H column. The enzyme protein contents were determined by the total Kjeldahl nitrogen analysis after trichloroacetic acid (TCA) precipitation.

Pretreated solids were hydrolyzed using 2.5, 5, 10 and 40 FPU (4, 8, 16 and 64 mg protein)/g glucan of Cellic Ctec2 at 50° C. and 200 rpm for 72 hrs. Substrates were suspended in 0.05M citrate buffer (pH 4.8) containing 1 g/L of sodium azide at 1% (w/v) glucan concentration. Hydrolyses were run in duplicates. Substrate and enzyme blanks were also run in parallel.

Scanning Electron Microscopy (SEM):

The surface of pretreated solids was imaged using a FESEM instrument with an Everhart Thornley detector. The FESEM imaging was performed at 5 kV and images were taken at magnifications ranging from 1000× to 40000×. The air-dried samples were mounted on aluminum stubs using carbon tape and then sputter coated with AuPd in the presence of argon gas with a Hummer I sputter coater.

Lignin Isolation:

Lignins were prepared by the extensive hydrolysis of carbohydrates of 4 hardwoods pretreated at 4 different severities. The pretreated hardwood chips were cryo-ground using liquid nitrogen and screened through the sieve with 0.84 mm square openings. Those were hydrolyzed with 80 FPU/g glucan of Spezyme CP and 160 CBU/g glucan of Novozyme 188 for 7 days. After the first hydrolysis, the pretreated solid of severity log Rₒ=10.44 was washed with DI water and incubated with 80 FPU cellulase/g glucan again for an additional 7 days. The final glucose yield after the first or second hydrolysis was 90-96%. Lignin residues were further cleaned to desorb the remaining cellulases on lignin surface. Lignin residue in DI water (2%, w/w) was sonicated for 60 min and washed with phosphate buffer (pH 7.0), and then treated with 0.1 ml protease/g dry solid in 0.05M phosphate buffer (2%, w/v) at 50° C. and 200 rpm for 24 h. Finally, the lignins were heated to 80-90° C. for 30 min for protease inactivation followed by washing with 1M NaCl solution and 3 times with DI water.

Differential Scanning Calorimetry (DSC):

Glass transition temperatures (T_{g}) of isolated lignins were measured using heat-flux DSC (Jade DSC, Perkin Elmer, Waltham, Mass.) equipped with Pyris software. A weight of 7-8 mg of air-dried lignins was pressed in a standard aluminum pan for a good thermal contact with a heating plate. A nitrogen flow of 20 ml/min was kept constant during the test. The measurement was run between 0 and 220° C. at a constant heating rate of 10° C./min. Each sample was heated to 220° C. and held there for 5 min to eliminate the thermal history. Lignins were cooled to 0° C. and then heated to 220° C. again. Data was provided from the second heating scan and measurements were repeated for three times. T_{g} was calculated as a mid-point of heat capacity changes.

Fourier Transform Infrared Spectroscopy (FTIR):

Attenuated total reflection fourier transform infrared (ATR-FTIR) spectra of lignins were obtained by using a Thermo-nicolet FTIR (Nexus 470) with OMNIC software. Spectra of each sample ranging from 4000 to 800 cm⁻¹ were averaged from 128 scans at a spectral resolution of 4 cm⁻¹. Relative absorbance for each band was calculated as the ratio of the band intensity of different groups to that of C—H vibration of the aromatic ring at 1505 cm⁻¹. The absorbance intensity was determined as the peak height and the baseline of spectra was automatically corrected.

Lignin Blocking Effect by Bovine Serum Albumin (BSA) on Enzymatic Hydrolysis:

Enzymatic hydrolysis of pretreated solids or Avicel in the presence of isolated lignins was performed with and without BSA (Sigma, St. Louis, Mo.). The substrates were pre-incubated with 50 mg BSA/g dry solid for 1 h at 25° C. and 200 rpm. After the pre-incubation, Cellic Ctec2 of 5 FPU (8 mg protein)/g glucan was added to the reaction mixture and further incubated for 72 hrs at 50° C. and 200 rpm.

Changes in Lignin Composition of Liquid Hot Water Pretreated Hardwood:

In order to understand the fate of lignin during the LHW pretreatment, the lignin content of pretreated hardwood was analyzed following NREL LAP standard procedures (Sluiter et al., 2008) (Table 4). The percentage of lignin increased from 29.3 to 40.3% with increasing pretreatment severity mainly due to the solubilization of xylan. In the range of pretreatment severity of log Rₒ=8.25-12.51, 75 to 85% of the lignin initially present was recovered in pretreated solids. However, at the severity of log Rₒ>11.39, the apparent lignin recovery increased to 90%. The higher pretreatment severity led to an increase in lignin recovery yield which might be caused by the condensation reactions of lignin with other degradation products.

TABLE 4
Lignin content changes during pretreatment

| | | | | | | | Total | Lignin |
|---|---|---|---|---|---|---|---|---|
| Sample | Severity | Temp. | Time | AIL^{a} | ASL^{b} | AIL/ASL | lignin^{c} | recovery^{d} |
| no. | factor | (° C.) | (min) | (%) | (%) | ratio | (%) | (%) |
| Untreated | | | | 28.1 | 3.8 | 7.4 | 31.8 | |
| 1 | 8.25 | 180 | 5 | 25.5 | 3.9 | 6.5 | 29.3 | 86.5 |
| 2 | 8.55 | 180 | 10 | 26.6 | 3.5 | 7.6 | 30.1 | 85.1 |
| 3 | 8.73 | 180 | 15 | 30.3 | 3.2 | 9.5 | 33.4 | 84.8 |
| 4 | 9.20 | 190 | 5 | 28.5 | 3.4 | 8.4 | 31.9 | 85.8 |
| 5 | 9.50 | 190 | 10 | 30.2 | 3.1 | 9.7 | 33.2 | 83.2 |
| 6 | 9.67 | 190 | 15 | 31.7 | 3.0 | 10.6 | 34.7 | 83.4 |
| 7 | 10.14 | 200 | 5 | 29.0 | 3.7 | 7.8 | 32.7 | 76.1 |
| 8 | 10.44 | 200 | 10 | 33.0 | 3.2 | 10.3 | 36.2 | 83.9 |
| 9 | 10.62 | 200 | 15 | 34.1 | 3.1 | 11.0 | 37.1 | 81.1 |
| 10 | 11.08 | 210 | 5 | 32.9 | 3.4 | 9.7 | 36.2 | 81.5 |
| 11 | 11.39 | 210 | 10 | 34.1 | 3.3 | 10.3 | 37.4 | 83.7 |
| 12 | 11.56 | 210 | 15 | 36.9 | 3.0 | 12.3 | 39.9 | 91.2 |
| 13 | 12.51 | 220 | 15 | 37.6 | 2.7 | 13.9 | 40.3 | 90.1 |

^{a}Acid insoluble Klason lignin
^{b}Acid soluble Klason lignin
^{c}Total lignin (%) = {Acid insoluble lignin (g)/total biomass (g) × 100} + Acid soluble lignin (%)
^{d}Lignin recovery (%) = Lignin recovered in solid after pretreatment (g)/Initial amount of lignin (g) × 100

While most of the initial lignin was retained in pretreated solids, changes in the ratio of acid insoluble lignin (AIL) to acid soluble lignin (ASL) were observed. When the pretreatment time increased at the given temperature, the content of AIL increased while that of ASL decreased. The AIL/ASL ratio increased from 6.5 to 13.9 as the pretreatment severity changed from log Rₒ=8.25 to 12.51. The increase in the AIL/ASL ratio indicates that lignin changes in its chemical composition or structure during pretreatment and is consistent with the literature. The AIL and ASL takes into account the quantification of total lignin as typically analyzed based on the Klason lignin method where hydrolysis in 72% sulfuric acid and boiling in 3% sulfuric acid separates lignin by dissolution of carbohydrates (Sluiter et al., 2008; Yasuda et al., 2001). During this process, lignin is partially solubilized in filtrate as ASL while AIL is separated as insoluble material. According to Yasuda et al. (2001), ASL may represent for the low-molecular-weight and hydrophilic derivatives of lignin.

The literature also shows that the content of methoxyl groups and the syringyl/guaiacyl (S/G) ratio of lignin in ASL were higher than those of AIL (Yasuda and Hirano, 1990). ASL was preferentially derived from the condensed syringyl lignin while the guaiacyl lignin was insoluble in 72% sulfuric acid. Based on the previous studies of sulfuric acid lignin, the structural properties of lignin including the S/G ratio might be changed during LHW pretreatment as shown in the increase of the AIL/ASL ratio. Samuel et al. (2010) showed that the S/G ratio of switchgrass lignin decreased from 0.80 to 0.53 after acid pretreatment when the AIL/ASL ratio increased. The changes from syringyl-guaiacyl (S-G) type lignin to a syringyl (S)-deficient type lignin with the increasing the pretreatment severity has been observed in the hydrothermal pretreatment studies as well (Chua and Wayman, 1979b; Jakobsons et al., 1995).

Morphological Changes of Pretreated Solids:

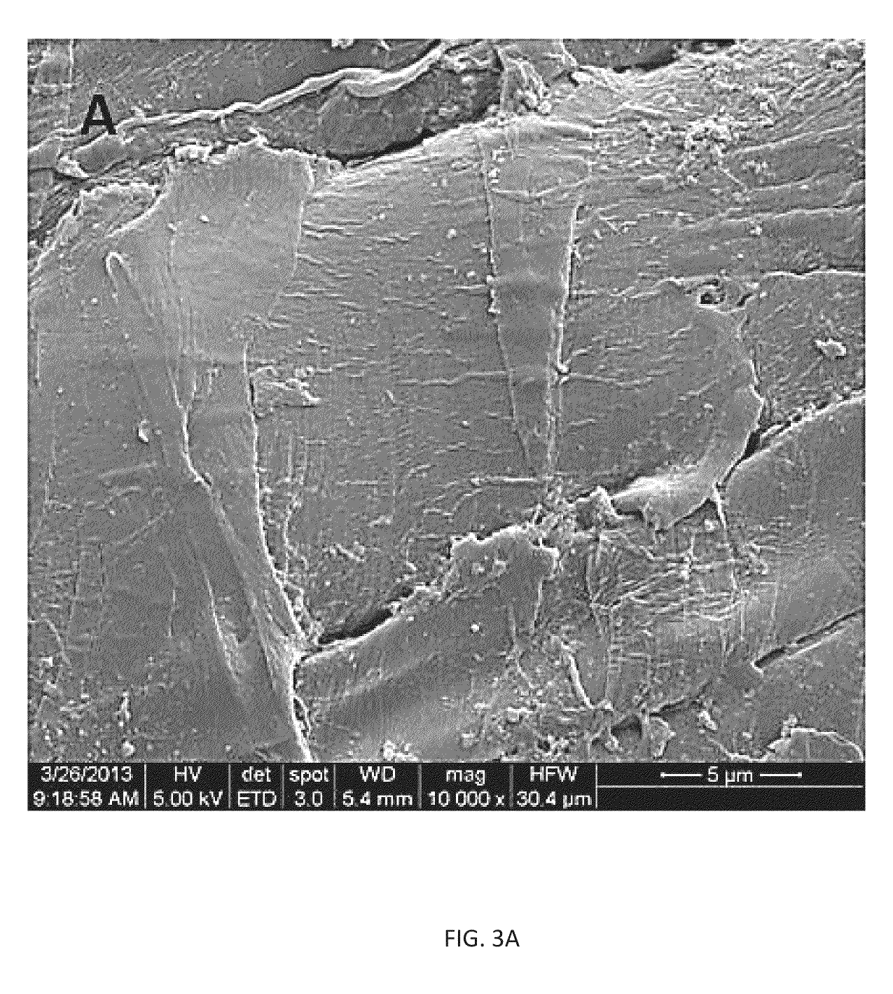

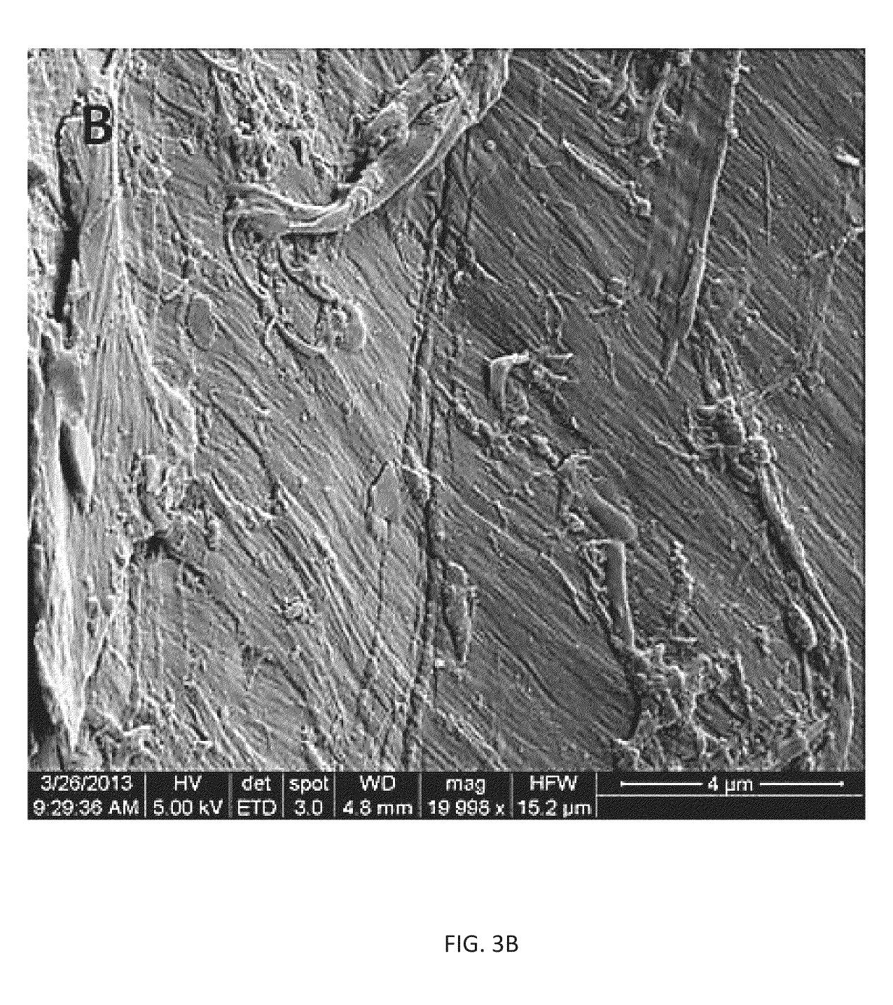

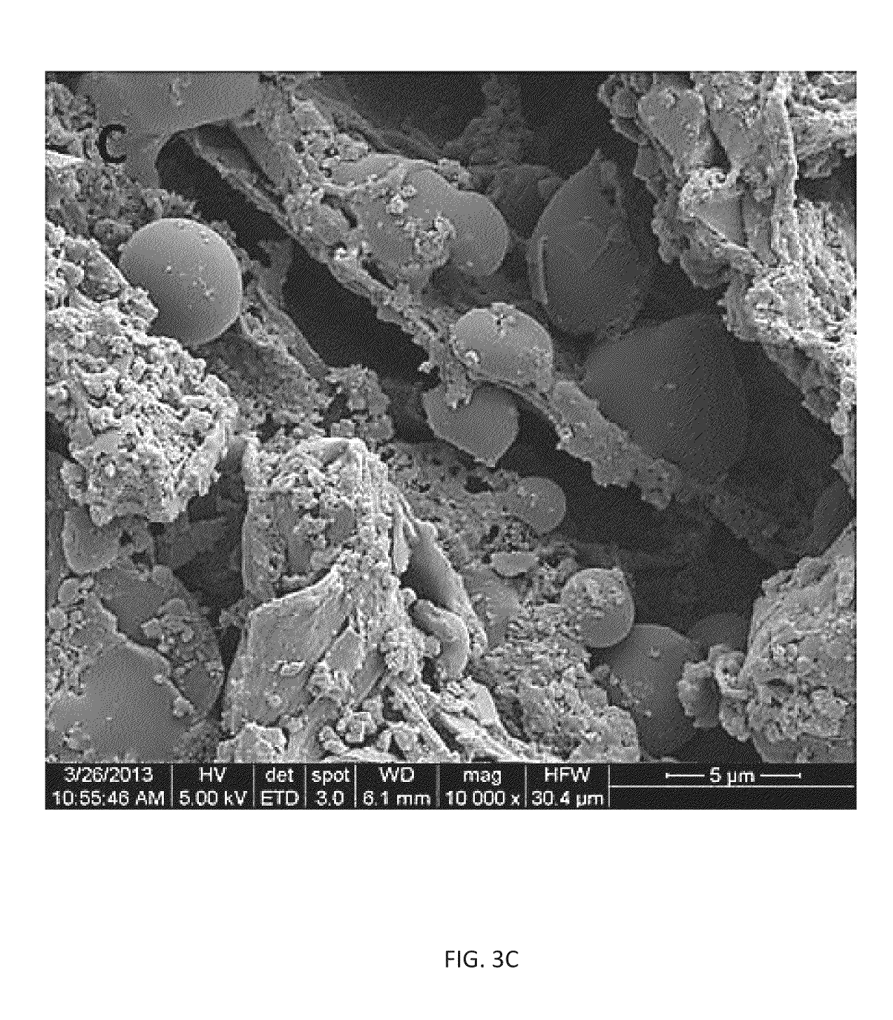

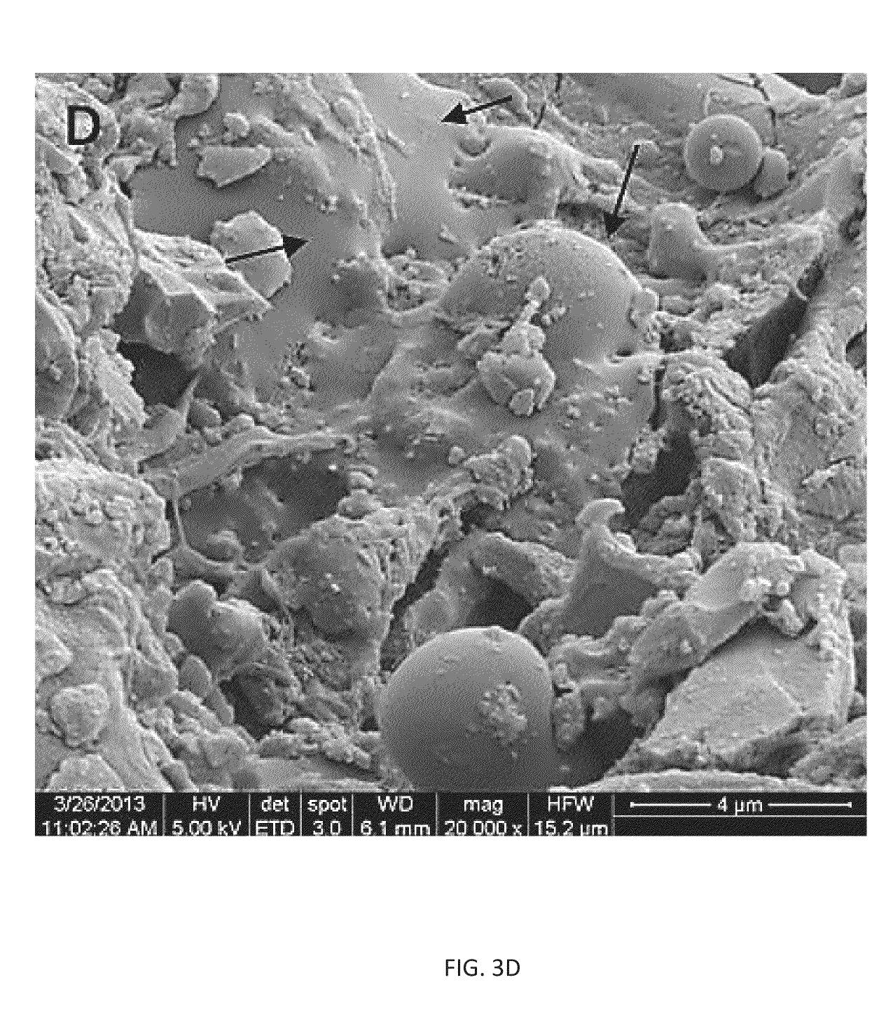

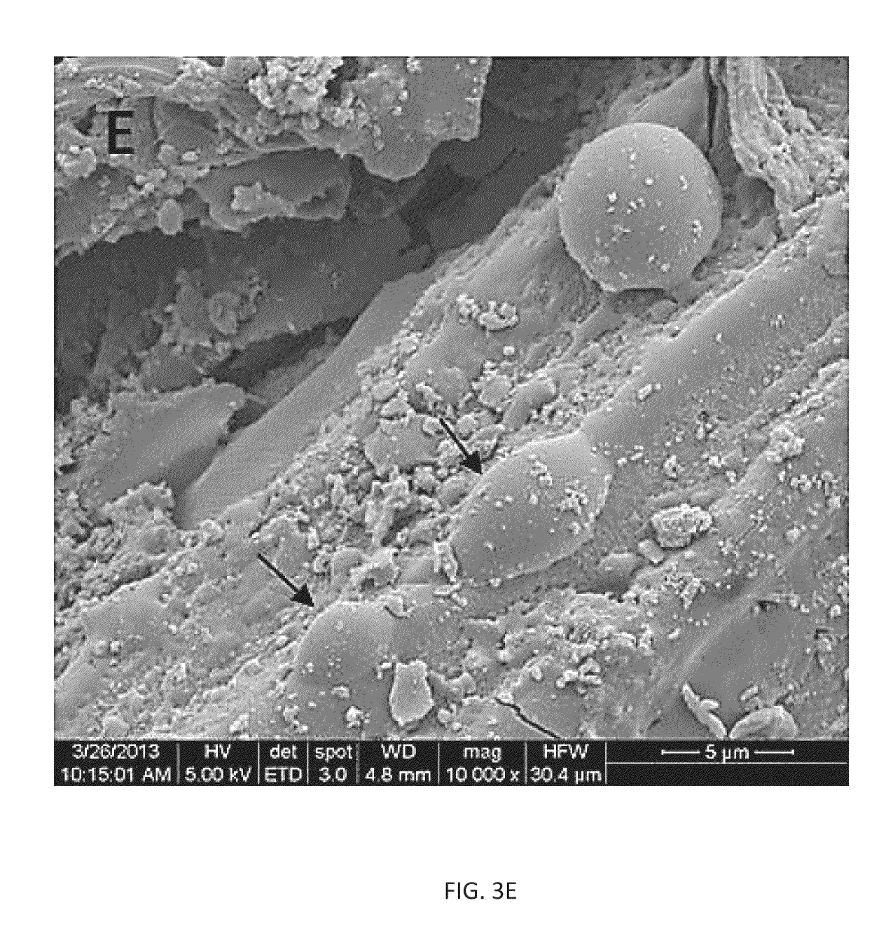

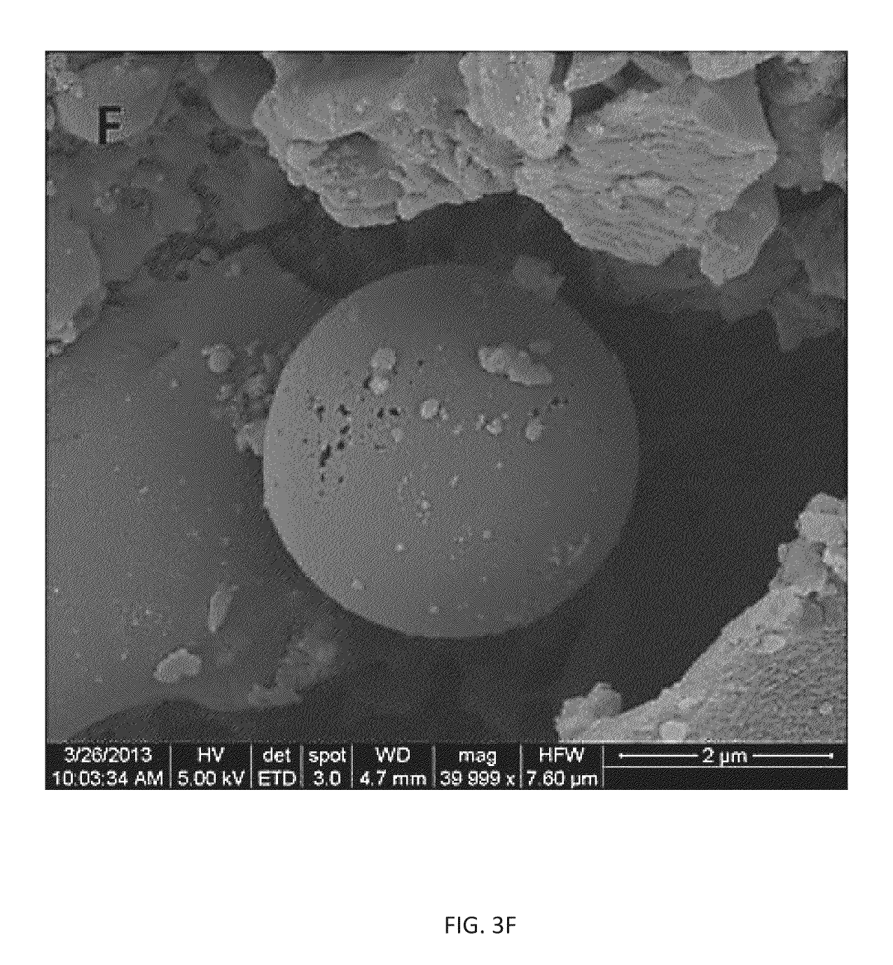

The morphological changes of LHW pretreated hardwoods were indicated by SEM imaging (FIGS. 3A-3F). Compared to the flat and smooth surface of untreated wood (FIGS. 3A and 3B), those of pretreated woods were disrupted and fragmented (FIGS. 3C-3F). The most noticeable change in pretreated wood was the appearance of spherical droplets on the surface while those were not found in untreated wood. The droplets were also observed in the corners of the disrupted cell walls therefore might act as a physical barrier inhibiting the access of enzymes to the inner region of cell walls (FIGS. 3C and 3F). FIGS. 3D and 3E show that the droplets, at least partially, were originated from the lignocellulosic matrix during pretreatment. The texture indicated by the arrows looks different from that of the original surface since some materials melted and re-solidified on the surface. The droplet formation in progress was also observed.

The spherical droplets found in the pretreated hardwood are thought to be mainly composed of lignin based on previous reports by Selig et al. (2007) and Donohoe et al. (2008). The hydrophobic lignin, which was melted and repolymerized during pretreatment, tends to form droplets to minimize their exposed surface in contact with aqueous solution. It has been suggested that the formation or redistribution of lignin droplets on cell walls usually occurs during hydrothermal pretreatment (Donohoe et al., 2008; Hansen et al., 2011; Selig et al., 2007). Donohoe et al. (2008) employed several techniques such as FTIR, NMR analysis, antibody labeling, and cytochemical staining in order to analyze spherical droplets formed from pretreated corn stover. They hypothesized that lignin melts and migrates through the cell wall during the pretreatment and redeposits on the surface upon cooling. Pseudo-lignin derived from the dehydrated carbohydrates during severe hydrothermal pretreatment was also suggested to be responsible for the formation of droplets (Hu et al., 2012; Kumar et al., 2013; Sannigrahi et al., 2008). The SEM images taken in this study show that the spherical droplets, probably composed of lignin or lignin-like materials (pseudo lignin), were found in LHW pretreated wood materials consistent with previously reported results for corn stover and wheat straw.

Enzymatic Hydrolysis of Pretreated Solids

LHW pretreated hardwoods were enzymatically hydrolyzed using 2.5, 5, 10 and 40 FPU (4, 8, 16 and 64 mg protein)/g glucan of Cellic Ctec2. At high enzyme loading (40 FPU), the glucose yield increased from 6.1 to ˜70% as the severity increased to log Rₒ=10.62. However, when the enzyme loading was reduced to 5 and 2.5 FPU, the glucose yield was less than 20 or 10%, respectively and did not show the yield improvement with increasing the severity. This phenomenon implies that there is a significant inhibitory effect of lignin on enzyme activities which shows in a companion paper to be due to non-productive adsorption at low enzyme loading. Also, enzymatic hydrolysis showed a plateau at high severities of log Rₒ=10.62-11.56 even though the xylan content was minimized. This may reflect increased lignin content, changes in lignin properties or physical hindrance of spherical droplets. As shown in SEM images, more lignin surface is exposed and the presence of spherical droplets deposited on the solid surface or in the cell wall corners can potentially inhibit the enzyme access to cellulose (Selig et al., 2007).

Lignin Isolation and Characterization:

To better understand changes in the lignin properties which might result from LHW pretreatment, isolated lignins were prepared from four different hardwoods pretreated at severities (calculated per Kim et al., 2013) of log Rₒ=10.44, 11.39, 11.56 and 12.51. Lignins were isolated by using the extensive enzymatic hydrolysis to remove carbohydrates from lignocelluloses. Table 5 summarizes the compositions of isolated lignins. The purity of the isolated lignins was obtained at 81.8-97.4%. While AIL increases from 79.6 to 95.4% according to the severity, the ASL content remained as 2.0%. It should be noted that a fraction of the lignin might become water-soluble during the extensive enzymatic hydrolysis (Nakagame et al., 2010, 2011b).

TABLE 5
Compositions of isolated lignins

| | | | | | | Total |
|---|---|---|---|---|---|---|
| | Temp. | Time | Glucan | AIL^{a} | ASL^{b} | lignin^{c} |
| Severity | (° C.) | (min) | (%) | (%) | (%) | (%) |
| 10.44 | 200 | 10 | 9.9 | 79.6 | 2.2 | 81.8 |
| 11.39 | 210 | 10 | 5.8 | 85.0 | 2.1 | 87.1 |
| 11.56 | 210 | 15 | 4.3 | 91.7 | 2.0 | 93.6 |
| 12.51 | 220 | 15 | 2.8 | 95.4 | 2.0 | 97.4 |

^{a}Acid insoluble Klason lignin
^{b}Acid soluble Klason lignin
^{c}Acid insoluble lignin + Acid soluble lignin

Glass Transition Temperature of Isolated Lignins

Since the glass transition temperature is sensitive to the structure of polymer, we examined the structural changes of lignin with T_{g} as an indicator. The rapid increase in heat capacity (Cₚ) curve (not shown) as a function of temperature suggests that lignin molecules go through a transition from the glassy state into the rubbery state. The T_{g} value of isolated lignins increased from 171 to 180° C. as the pretreatment severity increased from log Rₒ=10.44 to 12.51 (Table 6). The glass transition takes place in a broad temperature range between 150 and 188° C. for the isolated lignins of log Rₒ=10.44 and 11.39. For the highest severity lignin (log Rₒ=12.51), the drastic changes in Cₚ started to occur at 160° C. and ended at 205° C. The shifted and broadened T_{g} ranges at the higher severity indicates that lignins have more condensed and heterogeneous structures as severity increases. During hydrothermal pretreatment, the depolymerized lignin by the cleavage of ether linkages undergoes condensation reactions with the formation of C—C bonding which is more rigid and less reactive (Funaoka et al., 1990; Li et al., 2007). The condensation stiffens the lignin structure thus leading to the increase in T_{g}. Pan and Sano (2000) also found that the T_{g} increased as the extent of lignin condensation increased.

TABLE 6
Glass transition point and ranges of
isolated lignins

| Severity | T_{g} (° C.) | Range (° C.) |
|---|---|---|
| 10.44 | 171.9 ± 0.1 | 150-188 |
| 11.39 | 170.8 ± 0.1 | 150-185 |
| 11.56 | 174.2 ± 0.5 | 155-188 |
| 12.51 | 179.7 ± 0.1 | 160-205 |

Besides the degree of condensation of lignin, factors that might affect T_{g} include the functional groups, intermolecular bonding (cross-link) type, etc. (Glasser, 2000; Hatakeyama and Hatakeyama, 2004). Cross-links increase T_{g} by restricting the molecular motion of lignins. The T_{g} of water-saturated in situ hardwood lignin was reported to be 65-85° C., while that of softwood lignin was 90-105° C. (Olsson and Salmen, 1992). The lower degree of cross-links within hardwood lignin might lead to lower T_{g} when compared to softwood lignin. In a similar manner, the increased cross-links in lignin as a result of heating in acidic conditions exhibited the higher T_{g} than the untreated lignin (Olsson and Salmen, 1992). T_{g} was also shown to be dependent on the presence of certain functional groups such as methoxyl or hydroxyl groups which impart a flexibility to lignin. Hatakeyama and Hatakeyama (2004) revealed that T_{g} of methylated lignin decreased when its methoxyl content increased. It has been suggested that the methoxyl groups reduce the highly stable cross-links between aromatic units such as 5-5 and β-5 linkages, thereby resulting in a more flexible network (Kishimoto et al., 2010; Stewert et al., 2009).

The reported T_{g} values of most lignins were found to vary broadly from 80 to 200° C. (Glasser et al., 1983; Kelley et al., 1987; Kubo and Kadla, 2005; Olsson and Salmén, 1992; Schimidl, 1992). Even if lignins were prepared from the same plant species, they had considerably different T_{g} values depending on the isolation or pretreatment method used (Glasser, 2000). For example, the T_{g} value of lignin obtained from acid hydrolyzed aspen was 95° C. while that of steam explosion pretreated lignin was 139° C. (Glasser et al., 1983). In this study, the different T_{g} values depending on the severity indicate that the pretreatment alters the lignin structure. The lignin melts during LHW pretreatment above T_{g} (170-180° C.) and redeposits in the form of spherical droplets on the hardwood surface. Lignin derived from LHW pretreatment has characteristics that are analogous to lignins from other pretreatments.

Fourier Transform Infrared Spectroscopy (FTIR):

The FTIR analysis of isolated lignins was employed to follow the functional group changes such as methoxyl and aliphatic hydroxyl groups during LHW pretreatment. The spectra are presented in FIGS. 3A-3F, and the corresponding peak assignments based on the literature are summarized in Table 7. A broad band at 3350 cm⁻¹ attributed to aliphatic hydroxyl group decreased with increasing the pretreatment severity. The intensity of the primary hydroxyl groups at 1030 cm⁻¹ also decreased. Similar to our result, the content of aliphatic hydroxyl groups of dilute acid pretreated switchgrass lignin decreased compared to that of untreated lignin (Samuel et al., 2010). The intensity of methoxyl group at 1425 cm⁻¹ also showed a decreasing tendency along with the severity. The greater hydrolysis of methoxyl group at higher severity can be attributed to the degradation of aromatic rings and breakage of ether linkages.

TABLE 7
Relative absorbance of functional groups of isolated lignins

| | Band | | Severity of isolated lignins | | | |
|---|---|---|---|---|---|---|
| | (cm⁻¹) | Assignment | 10.44 | 11.39 | 11.56 | 12.51 |
| 1 | 3350 | Aliphatic OH stretch | 0.55 | 0.47 | 0.45 | 0.34 |
| 2 | 3000 | C—H stretch | 0.24 | 0.24 | 0.24 | 0.20 |
| 3 | 2840 | C—H stretch | 0.08 | 0.10 | 0.11 | 0.07 |
| 4 | 1595 | C—H vibration + C—O stretch | 0.99 | 0.96 | 1.00 | 1.00 |
| 5 | 1505 | C—H vibration | 1.00 | 1.00 | 1.00 | 1.00 |
| 6 | 1455 | C—H deformation (CH₂, CH₃) | 0.84 | 0.80 | 0.73 | 0.60 |
| 7 | 1425 | OCH₃ | 0.61 | 0.56 | 0.54 | 0.44 |
| 8 | 1320 | C—H vibration with C—O stretch of S-ring | 0.50 | 0.50 | 0.44 | 0.33 |
| 9 | 1260 | C—O of uncondensed G-ring | 0.11 | 0.12 | 0.12 | 0.10 |
| 10 | 1210 | C—H vibration with C—O | 0.96 | 1.04 | 0.86 | 0.91 |
| | | stretch | | | | |
| 11 | 1100 | C—O deformation in aliphatic ether | 1.48 | 1.45 | 1.28 | 1.17 |
| 12 | 1030 | C—H deformation in uncondensed G-ring | 1.08 | 0.89 | 0.60 | 0.50 |
| | | with C—O deformation in 1ry OH | | | | |

Each band was assigned according to Chua and Wayman (1979b), Hergert (1971), Kubo and Kadla (2005), Nada et al. (1998), Nakagame et al. (2011b) and Pandey (1998). Data is the average of triplicate analysis.

Chua and Wayman (1979b) showed that the content of methoxyl group of autohydrolyzed hardwood lignin decreased as the cooking time increased. The de-methoxylation of the lignin during autohydrolysis is considered to account for the variation of the distribution of syringyl and guaiacyl units. Therefore, the decrease in the intensity of methoxyl group may imply that lignin of higher severity is tending toward the syringyl-deficient softwood lignin structure. The decreasing intensity of the 1320 cm⁻¹ band (C—H vibration with C—O stretch of syringyl unit) was also observed.

The decrease in both hydroxyl and methoxyl groups explains the increase in T_{g} at higher severity. As also shown in the increase of the AIL/ASL ratio, lignin structure is assumed to be changed to the more condensed and less reactive type during pretreatment. The changes in lignin structure were further studied by evaluating the degree of inhibition on enzyme activities as presented in the following section.

Inhibitory Effect of Isolated Lignin on Avicel Hydrolysis

In this study, the isolated lignins were separately loaded to the enzyme-cellulose reaction mixtures so that the main inhibitory mechanism of lignin would be the non-productive adsorption of enzymes onto lignin. In order to evaluate the inhibitory effect of isolated lignins on the cellulose hydrolysis, 5 FPU (8 mg protein)/g glucan of Cellic Ctec2 was incubated with 1% (w/v) of Avicel in the presence of 0.5% (w/v) of lignin. The enzymatic hydrolysis time course indicated that the presence of isolated lignins reduced the hydrolysis yield as well as the initial hydrolysis rate. When the isolated lignin of severity log Rₒ=10.44 was added, the glucose yield decreased from 62.5 to 58%. For lignin obtained from higher severities of log Rₒ=11.39-12.51, the glucose yield was reduced to 51%. In the previous studies by Nakagame et al. (2011b), lignins isolated from softwoods pretreated at higher severities had a negative effect on Avicel hydrolysis as well.

Lignin Blocking Effect by BSA on Enzymatic Hydrolysis:

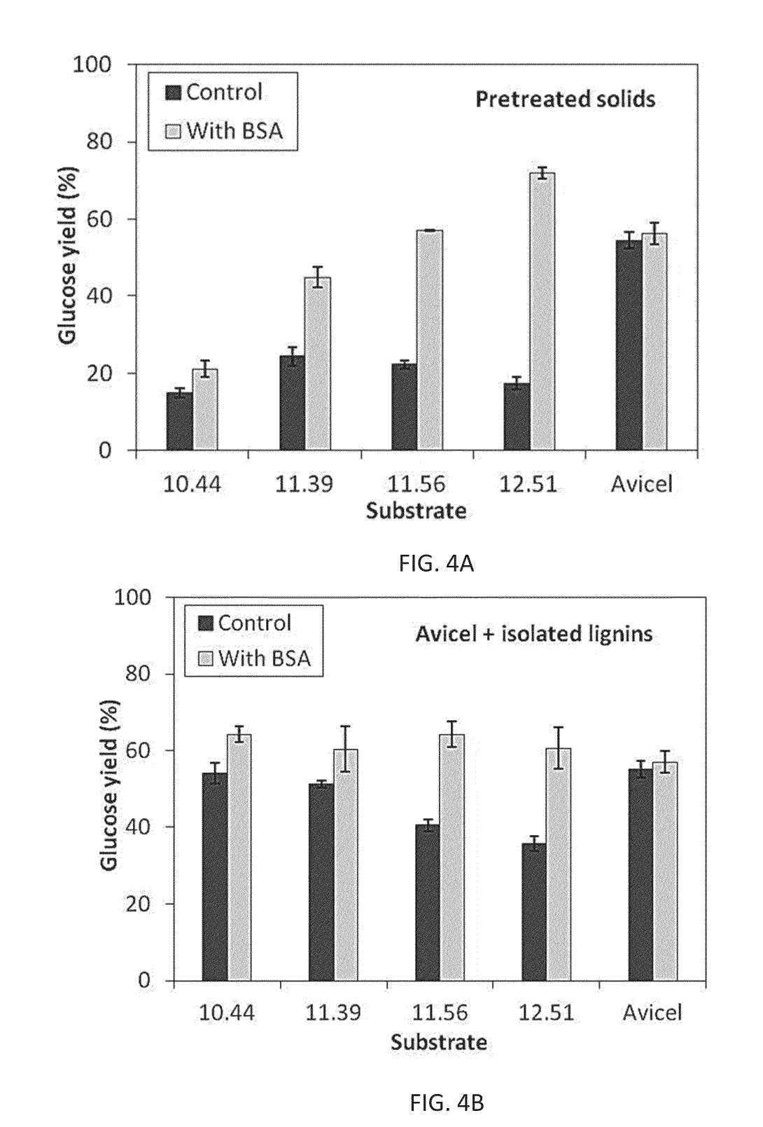

To investigate the inhibitory role of lignins on cellulose hydrolysis, additives such as surfactants and non-catalytic protein (BSA) have been used as lignin surface blockers in many papers (Eriksson et al., 2002; Kristensen et al., 2007; Kumar et al., 2012; Sipos et al., 2010; Yang and Wyman, 2006). The additives were hypothesized to prevent the non-productive adsorption of enzymes onto lignin, thereby resulting in the improved enzymatic hydrolysis efficiency. In this study, hardwoods pretreated at different severities of log Rₒ=10.44-12.51 were pre-incubated with 50 mg of BSA per gram dry solid before the hydrolysis at low enzyme loading of 5 FPU (8 mg protein)/g glucan (FIG. 4A). The lignin content of pretreated hardwoods ranged from 36.2 to 40.3%. The improvement in the enzymatic hydrolysis yield by the addition of BSA increased significantly as the pretreatment severity increased. At the highest severity of log Rₒ=12.51, the enzymatic hydrolysis yield increased from 17.4 to 71.9% in the presence of BSA. This indicates pretreatment causes changes in the lignin and other cell wall structures resulting in non-productive adsorption of enzyme. Further characterization of this effect is needed to better understand the interactions between enzymes and biomass.

Enzymatic hydrolysis of Avicel with isolated lignins was performed at equivalent cellulose to lignin ratios so that glucan (Avicel) was 55-58% and lignin was 36-40% (FIGS. 4A and 4B). The final concentration of Avicel and lignins loaded into the reaction vessel was 1% (w/v) and 0.65-0.70% (w/v), respectively. The enzymatic hydrolysis yield of Avicel decreased gradually from 54 to 36% depending on severity of pretreatment, thus indicating that lignin plays a major role in decreasing extents of hydrolysis with pretreatments of increasing severity.

Substrate features that might affect the enzymatic hydrolysis were investigated for the lignin fraction of the hardwood cell wall structure. The increased content of lignin in hardwood as well as the changes in lignin structure during LHW pretreatment reduces enzymatic hydrolysis of cellulose. While the glucose yield at high enzyme loading (40 FPU (64 mg protein)/g glucan) hydrolysis increased from 6.1 to 70% as severity increased from 8.25 to 10.62, hydrolysis at low enzyme loading (<5 FPU (8 mg protein)/g glucan) was less than 20% and an increase in the glucose yield was not observed at higher severities of log Rₒ>10.62. While non-productive adsorption of enzyme onto lignin surface is considered to be a major inhibitory mechanism on enzymatic hydrolysis, a disproportionate decrease occurs when enzyme loading is decreased.

Adsorption of Enzyme onto Lignins of Liquid Hot Water Pretreated Hardwoods:

Enzymes:

Cellic Ctec2 (118 FPU/mL, 190 mg protein/mL) was provided by Novozyme, North America Inc. (Fran. Novozyme 188 with the protein content of 152 mg/mL was purchased from Sigma.

Pretreatment and Lignin Isolation:

Mixed hardwood chips were liquid hot water (LHW) pretreated at 200-220° C. for 10-15 min according to the previously described procedures. The pretreatment condition was expressed as a severity factor, log Rₒ (Rₒ=t×exp ((T−100)/ω) (Overend and Chornet, 1987) and the value ω=4.6 from Kim et al. (2013) was used. Isolated lignins were prepared from 4 LHW pretreated hardwoods of severities log Rₒ=10.44, 11.39, 11.56 and 12.51. Lignins were isolated by using excessive amount of cellulase and protease as described in Ko et al. (2014). The nitrogen content of final lignin was measured by Kjeldahl nitrogen analysis in duplicate. The final lignins were kept at 4° C. until use.

Brunauer, Emmett, and Teller (BET) Surface Area:

BET surface areas of isolated lignins were measured using a Micromeritics TriStar II 3020 at Micromeritics Analytical Services (Norcross, Ga.). Samples were dried under vacuum at 40° C. overnight before analysis and the surface area was determined by nitrogen adsorption according to the multi-point BET procedure. Data was obtained by a single measurement.

Adsorption Experiments:

The adsorption isotherm of enzyme on isolated lignins was studied with varying the protein concentrations from 25 to 2,000 μg/ml (2.5-200 mg protein/g dry lignin). Lignins and enzymes were incubated in a hybridization incubator (FinePCR, GyeongGi, Korea) with rotating mixing (20 inversions per min) at 25° C. for 1.5 hrs to reach equilibrium. The adsorption was carried out in a total volume of 500 μL using 1.5 mL-Eppendorf tube which contained 10 mg/mL of substrate, enzymes and 0.05 M citrate buffer (pH 4.8). Lignins in the buffer were thoroughly dispersed by sonication for 10 seconds. After incubation, the supernatant was separated from the solid fraction by centrifugation at 13,000 rpm for 2 min. The centrifuged supernatant was further filtered through the low protein binding membrane (0.22 μm, Millex GV, Millipore). The concentration of non-adsorbed proteins in the supernatant was measured using a bicinchoninic acid (BCA) protein assay reagent kit (Thermo-Scientific, Rockford, Ill.). The adsorbed enzyme was calculated from the difference between the total initial protein amount and the non-adsorbed protein amount. All adsorption reactions were run in triplicate. Controls lacking lignins or enzymes were also run in parallel. Experimental adsorbed protein data was fitted into the following Langmuir equation (Equation (2)):

$${E}_{ads} = \frac{Emax \cdot K \cdot Efree}{1 + K \cdot Efree} \qquad ( 2 )$$

Where E_{ads} is the amount of adsorbed enzyme (mg/g lignin), E_{free} the amount of free (non-adsorbed) enzyme in supernatant (mg/mL), Eₘₐₓ the maximum amount of adsorbed enzyme (mg/g lignin), and K the Langmuir constant (mL/mg enzyme). The constants were determined by non-linear regression of experimental data using Excel Solver program (Kemmer and Keller, 2010).

Enzyme Activity Assays:

The endo-glucanase (EG) activity was assayed using 1% (w/v) carboxymethyl cellulose (CMC, Sigma-aldrich, St. Louis, Mo.) as substrate. The mixture containing 450 μL of 1% CMC and 50 μL of the supernatant (enzyme sample) was incubated at 50° C. for 10 min. The reaction was stopped by adding 2.0 mL of DNS reagent and boiled at 95° C. for 5 min. The released reducing sugars were measured according to the DNS curve with glucose as standard. The cellobiohydrolase (CBH) activity was estimated by its ability to hydrolyze the agluconic bond (between the p-nitrophenol and cellobioside) of p-nitrophenyl-β-D-cellobioside (pNPC). To inhibit β-glucosidase activity which also acts on the agluconic bond, 1 mM of D-glucono-1,5-lactone was added to the substrate solution (Deshpande et al., 1984). After the incubation at 50° C. for 10 min, 1.0 mL of cold 1M sodium carbonate was added and the optical density was measured at 410 nm. One enzyme unit is defined as the amount of enzyme releasing 1 μmol of p-nitrophenol per min under the specific condition. For measuring β-glucosidase activity, 10 mM of p-nitrophenyl-β-D-glucoside (pNPG) was used as substrate.

Protein Analysis by Electrophoresis:

Differential adsorption of enzyme components onto lignin was examined by sodium dodecyl sulfate polyacrylamide gel electrophoresis (SDS-PAGE). Lignin (2%, w/v) of severity log Rₒ=12.51 was incubated with Ctec2 at 40 mg protein/g lignin for 1.5 hrs at 25° C. The samples were prepared from the supernatant and loaded to precast 4-20% Tris-HCl gradient gel (Bio-Rad Laboratories Inc., Hercules, Calif.) and run using Mini-PROTEAN®II cell electrophoresis system. The gel was visualized by staining with Coomassie Blue G-250 (Bio-Rad). The supernatant fractions of enzyme and lignin controls were also run in parallel.

Profiles of Isolated Lignins:

Lignin-rich residues were isolated from four LHW pretreated hardwoods of different severities ranging from log Rₒ=10.44 to 12.51. The nitrogen content of lignin was measured to estimate the residual cellulases remaining in the lignins after the lignin isolation procedure (Table 8). While the nitrogen contents of pretreated solids were below 0.2%, those of isolated and buffer-washed lignins before protease treatment (1.44-2.50% nitrogen) were relatively high. This gives a first indication that cellulases adsorb irreversibly onto the lignins during the enzymatic hydrolysis. Protease treatment of lignin to which enzyme proteins had adsorbed reduced the nitrogen content by two- or three-fold resulting in 0.57-0.70% in the final lignins. This level of nitrogen content remaining in lignins is considered to be low enough not to affect the subsequent cellulase adsorption experiments as shown in the previous studies with lignins containing 0.6-1.1% of nitrogen (Kumar and Wyman, 2009; Nakagame et al., 2011).

TABLE 8
Measurement of specific surface area and
nitrogen content of isolated lignins

| | | | Specific | Nitrogen (%) | | |
|---|---|---|---|---|---|---|
| Severity | | | surface | | Lignin | Lignin |
| factor | | Pretreatment | area | Pretreated | before | after |
| Kim^{a} | OC^{b} | condition | (m²/g) | solids | protease | protease |
| 10.44 | 3.94 | 200° C., 10 min | 16.4 | 0.20 | 2.50 | 0.70 |
| 11.39 | 4.24 | 210° C., 10 min | 18.3 | 0.18 | 1.52 | 0.57 |
| 11.56 | 4.41 | 210° C., 15 min | 16.2 | 0.16 | 1.66 | 0.62 |
| 12.51 | 4.71 | 220° C., 15 min | 14.0 | 0.17 | 1.44 | 0.61 |

^{a}Severity factor with ω = 4.6 (Kim et al., 2013)
^{b}Conventional severity factor with ω = 14.75 (Overend and Chornet, 1987)

The specific surface area of isolated lignins was measured by using the BET method. Surface areas of lignin particles ranged from 14.0 to 18.3 m²/g (Table 8). At higher pretreatment severities above log Rₒ=11.39 (>210° C.), the specific surface area of lignins tended to decrease slightly. Compared to our results for hardwood lignins where specific surface area decreased, lignins isolated from steam pretreated softwood showed that the surface area increased from 34.2 to 64.4 m²/g when the pretreatment temperature increased from 190 to 200° C. (Nakagame et al., 2011). Rahikainen et al. (2011) observed that the surface areas of lignins varied considerably depending on the lignin preparation method. While the enzymatically isolated lignin had the surface area of 2.5 m²/g, that of lignins prepared by acid hydrolysis was 80 m²/g. The larger surface area of acid hydrolysis lignin was found to have the stronger inhibitory effect on Avicel hydrolysis than the enzymatically isolated lignin (Rahikainen et al., 2011).

Enzyme Adsorption Isotherms:

Adsorption isotherms were generated using isolated lignins incubated with different enzyme loadings (2.5-200 mg protein/g lignin) at 25° C. for 1.5 hrs. Adsorption parameters were estimated by fitting the adsorption data to the Langmuir equation. Table 9 shows the constants of the maximum adsorption capacity (Eₘₐₓ), affinity (K) and partition coefficient (Kₚ) for isolated lignins of different severities. The maximum adsorption capacities (Eₘₐₓ) of lignins varied from 36.6 to 44.8 mg protein/g lignin. The difference in the adsorption capacity depending on the severity was not as significant as given in the previous work by Ooshima et al. (1990). They reported that the maximum adsorption capacities of lignins isolated from dilute-acid pretreated hardwood decreased from 100 to 12.3 mg protein/g lignin as the pretreatment temperature increased from 180 to 220° C. The drastic decrease in adsorption capacity was attributed to the condensation of melted lignin molecules during pretreatment (Ooshima et al., 1990). We observed only a slight decrease in adsorption capacity at the highest severity (log Rₒ=12.51) in our result which may also reflect the condensed lignin structure having the lowest BET specific surface area. Similar to our result, lignins from softwood which was steam-pretreated at 190-210° C. showed no significant differences in adsorption capacity ranging from 59 to 71 mg protein/g lignin and had the highest value at 200° C. (Nakagame et al., 2011). The reported adsorption capacity values of lignins varied from 56.8 to 126.9 mg protein/g lignin and increased with respect to the pretreatment types using ammonia fiber expansion (AFEX), dilute acid, steam explosion and lime (Kumar and Wyman, 2009).

TABLE 9
Maximum cellulase adsorption capacity (Eₘₐₓ),
affinity (K) and partition coefficient (Kₚ)
constants for isolated lignins of different severities

| | Eₘₐₓ | K | Kₚ = Eₘₐₓ × K |
|---|---|---|---|
| Lignin | (mg protein/g lignin) | (mL/mg protein) | (mL/g lignin) |
| 10.44 | 37.0 | 2.8 | 105.1 |
| 11.39 | 44.8 | 2.9 | 130.2 |
| 11.56 | 44.5 | 3.0 | 132.9 |
| 12.51 | 36.6 | 4.9 | 178.2 |

The affinity (K) in the adsorption of enzyme onto lignins increased from 2.8 to 4.9 mL/mg protein when the pretreatment severity increased (Table 9). The strength of interaction between substrate and enzyme was also characterized by the partition (distribution) coefficient (Kₚ=Eₘₐₓ×K), which was derived from the initial slope of the Langmuir isotherm at low surface coverage (Kyriacou et al., 1988; Medve et al., 1997; Nidetzky et al., 1994). As the pretreatment severity increased, the values of partition coefficient also increased from 105.1 to 178.2 mL/g lignin (Table 9), showing that the interactions between lignins and enzymes get stronger at the higher severities.

The more condensed lignin structure has a higher glass transition temperature (T_{g}) and coincides with more extensive enzyme adsorption as severity increases. The structural changes of lignins into syringyl-deficient and softwood-like forms can explain the increasing values of the affinity for enzyme adsorption at higher severities. The increase in the adsorption affinity at higher pretreatment severities is in consistent with the previous work using lignin prepared from steam-exploded woods (Nakagame et al., 2011; Ooshima et al., 1990). As the pretreatment temperature increased from 190 to 210° C., the partition coefficient (binding strength) of softwood lignin increased from 151 to 266 mL/g lignin (Nakagame et al., 2011). Steam pretreatment is hypothesized to increase the degree of condensation and hydrophobicity of lignin, thereby resulting in adsorption of more enzyme via hydrophobic interaction.

Distribution of Adsorbed Enzyme Components onto Lignins:

Changes in lignin properties depend on pretreatment severity and affect the non-productive adsorption behavior of enzymes but may also result in preferential adsorption of one component over the other. The adsorption profile of cellulases was further investigated by analyzing the distribution of the adsorbed enzyme components. A commercial cellulase cocktail, Cellic CTec (Novozyme, North America Inc.), used in this study was derived from Trichoderma reesei containing at least the two main cellobiohydrolases (Cel7A and Cel6A) and five different endo-glucanases (Cel7B, Cel5A, Cel12A, Cel61A, and Cel45A) with a high level of f3-glucosidase (Pedersen et al., 2011). After the incubation of Cellic Ctec2 at 100 μg protein/mL with lignin, the remaining enzyme activities in the supernatant were measured to estimate the adsorption degree by difference. Cellobiohydrolase (CBH), endo-glucanase (EG), and β-glucosidase (β-G) activities were evaluated by using pNPC, CMC, and pNPG as substrate, respectively.

The remaining activity of EG in buffer was 49.6-59.2% while the total free protein was observed in the ranges of 42-62%. For CBH, the incubation with lignins resulted in the reduction of its original activity to 54-63%. As the severity of lignins increased, the degree of adsorption of EG was slightly lowered by 9.6% while that of CBH had no specific tendency. However, most of β-G activity was lost. The remaining activity of β-G in the supernatant decreased from 17.3 to 2.0%, for lignins isolated from hardwoods pretreated at increasing severities. A similar specificity of cellulase adsorption onto lignins was observed by Rahikainen et al. (2011) who showed 60% loss in EG and CBHI (Cel7A) activities and more than 80% loss in β-G activity when the commercial T. reesei cellulase (Celluclast) was incubated with the enzymatic hydrolyzed (lignin-rich) residue of steam exploded softwood (Rahikainen et al., 2011). The high level of β-G adsorption was also reported for lignins prepared from steam-pretreated hardwood. Sutcliffe and Saddler (1986) observed that β-G from Trichoderma harzianum was the most strongly affected component among cellulase enzymes adsorbed by lignin.

In contrast, β-G (Novozyme 188) from A. niger has been shown to be least affected among several enzymes including cellulases and xylanases for organosolv or steam pretreated softwood lignins (Berlin et al., 2006; Sipos et al., 2010) and interestingly is also least affected by soluble lignin inhibitors as well (Ximenes et al., 2010, 2011). The adsorption of β-Gs from two sources (Cellic Ctec2 and Novozyme 188) onto lignin from steam pretreated wheat straw was compared by Haven and Jørgensen (2013). Kumar and Wyman (2009) reported that the affinity constant of β-G (Novozyme 188) for lignins (K=0.01-0.09 mL/mg) was much lower than that of cellulase (K=0.11-2.14 mL/mg). Compared to the strong adsorption of T. reesei β-G, the loss of A. niger β-G activity was relatively low. The free β-G activities that remained in the supernatant were 63-76%. These results further confirm the β-G adsorption is related to the microorganism from which it is derived as suggested by Ximenes et al. (2010) and indicates protein engineering with directed evolution could result in β-G having lower affinity for lignin.

The free β-G activity from T. reesei was almost depleted in the supernatant due to the non-productive adsorption onto lignin whereas the loss of A. niger β-G activity was less severe. This might be due to the different β-G properties including different pIs (Table 10). A similar observation has also been reported with the deactivation or inhibition of enzymes by soluble phenolic components derived from lignin degradation products (Ximenes et al., 2010, 2011). Phenols deactivated β-G from T. reesei to a greater extent than that from A. niger and the deactivation rate constants by polymeric and monomeric phenols were compared in Table 10. This deactivation was a time-dependent process which might be resulted from the binding of phenolic inhibitors at the active site of enzyme (Ximenes et al., 2011).

TABLE 10
Properties of β-glucosidases from T. reesei and A. niger

| | Protein | M_{w} | Theoretical pI | k (h⁻¹) of | k (h⁻¹) of |
|---|---|---|---|---|---|
| Source | family | (kDa)^{a} | (Experimental)^{a} | tannic acid^{b} | gallic acid^{b} |
| T. reesei | GH3 | 78.4 | 6.38 (8.5) | 0.068 | 0.046 |
| A. niger | GH3 | 93.2 | 4.65 (4.55) | 0.045 | 0.006 |

^{a}Molecular weight (M_{w}) and theoretical pIs were from Uni-Prot. Experimental pIs were adapted from Chirico and Brown (1987) and Lima et al. (2013).
^{b}Deactivation rate constants of polymeric (tannic acid) and monomeric (gallic acid) phenols (Ximenes et al., 2011).

SDS-PAGE Analysis of Free T. reesei Cellulase Distribution

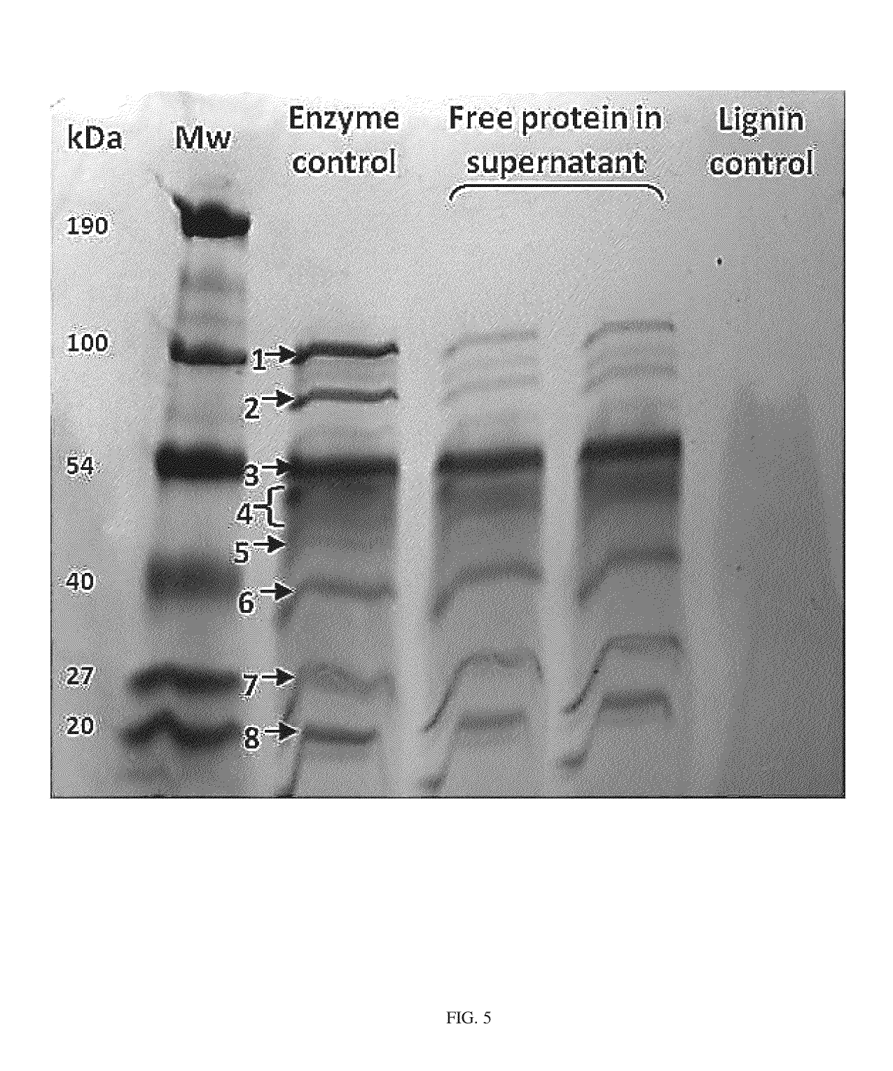

The distribution of cellulases in the supernatant (unbound fraction) was analyzed by SDS-PAGE and compared to activity tests (FIG. 5). A variety of enzymes that synergistically degrade lignocellulose have been identified in T. reesei (Foreman et al., 2003; Ouyang et al., 2006) and the enzymes corresponding to each band were specified in Table 11. In the SDS-PAGE analysis of samples, the two high molecular weight bands at 80-100 kDa (band 1&2) showed the strongest decrease in its intensity and corresponded to β-G. This was in accordance with the ˜98% loss of β-G activity in the supernatant due to the non-productive adsorption to lignin. Changes in major CBH and EG proteins (bands of 3-5) which lost ˜40% of its original activities were less clear due to their similar molecular weights (CBHI 54 kDa, CBHII 50 kDa, EGI 48 kDa, EGII 44 kDa). CBHI which was likely to be in band 3 seemed unchanged. Bands 4 and 5 were slightly narrowed and disappeared after incubation with lignin, respectively, indicating adsorption and a decrease in free activities.

TABLE 11
Profile of putative enzymes produced from T. reesei

| | Gene | Protein | Amino | M_{w} | Theoretical pI | | | Band |
|---|---|---|---|---|---|---|---|---|
| Enzyme | name | family | acids^{a} | (kDa)^{a} | (Experimental)^{b} | | Hydrophobicity^{c} | no. |
| CBHI | cel7a | GH7 | 513 | 54.1 | 4.65 | (3.6-3.9) | −0.437 | 3 |
| CBHII | cel6a | GH6 | 471 | 49.6 | 5.11 | (5.2-5.9) | −0.118 | 4 |
| EGI | cel7b | GH7 | 459 | 48.2 | 4.73 | (3.9-4.5) | −0.367 | 4 |
| EGII | cel5a | GH5 | 418 | 44.2 | 4.97 | (4.2-5.5) | −0.190 | 5 |
| EGIII | cel12a | GH12 | 234 | 25.2 | 6.69 | (6.8-7.4) | −0.226 | 7-8 |
| EGIV | cel61a | GH61 | 344 | 35.5 | 5.29 | | −0.065 | 6 |
| EGV | cel45a | GH45 | 242 | 24.4 | 4.21 | | −0.113 | 7-8 |
| β-GI | cel3a | GH3 | 744 | 78.4 | 6.38 | (8.5) | −0.163 | 2 |

| | | | | | | | |
|---|---|---|---|---|---|---|---|
| β-GX^{d} | cel3b | GH3 | 874 | 93.9 | 5.73 | −0.317 | 1 |
| XYNI | xyn11a | GH11 | 229 | 24.5 | 5.0 | −0.278 | 7-8 |
| XYNII | xyn11b | GH11 | 222 | 24.2 | 8.89 | −0.469 | 7-8 |
| XYNIII | xyn10a | GH10 | 347 | 38.1 | 6.97 | −0.211 | 6 |

^{a}Amino acid length and molecular weight (M_{w}) were from Uni-Prot (http://www.uniprot.org/).
^{b}Theoretical pI was computed using ExPASy ProtParam tool
(http://web.expasy.org/protparam/). Experimental pIs were adapted from Chirico and Brown (1987), Hui et al. (2001), Medve et al. (1998), Nakagame et al. (2011) and Vinzant et al. (2001).
^{c}GRAVY (Grand average of hydropathy index) value was used as an indicator of hydrophobicity of enzymes (Kyte and Doolittle, 1982). The more positive score indicates the greater hydrophobicity. The value was computed using a ExPASy ProtParam tool for a given protein stored in Uni-Prot.
^{d}Putative β-glucosidase reported by Foreman et al. (2003), Häkkinen et al. (2012) and Ouyang et al. (2006). β-GIII and IX are also candidates for 91-94 kDa.

As well as the structural and physicochemical characteristics of lignin itself, the enzyme properties including hydrophobicity, molecular size, electrical charge and structural stability can affect the adsorption (Norde, 1986, 1996). These properties of T. reesei enzymes are summarized in Table 11. The hydrophobicity of enzyme was estimated as a parameter, grand average of hydropathy index (GRAVY), based on the amino acid sequence and exhibited the different values. Among the cellulase (CBH and EG) structure, the carbohydrate binding module (CBM) has been considered to play an important role on hydrophobic interaction between lignin and cellulase (Borjesson et al., 2007; Palonen et al., 2004). The conserved aromatic residues on the flat surface of CBM such as tyrosine or tryptophan have shown to be responsible for the high adsorption affinity of cellulase to lignin (Borjesson et al., 2007; Rahikainen et al., 2013). Contrary to cellulase (CBH and EG), β-G carries no CBM and has an activity toward small soluble substrates but not on insoluble carbohydrate. However, A. niger β-G (116 kDa) has a cellulase-like tadpole structure consisting of catalytic domain (CD) and fibronectin III-like domain (FnIII) connected by a long linker region which is common for enzymes working on insoluble carbohydrates (Lima et al., 2013). It was suggested that FnIII domain causes β-G to adsorb onto lignin. Lima et al. (2013) argued that this tadpole-like structure might be common in GH3 fungal β-Gs based on the amino acid sequence comparison. This can partially explain the interaction between lignin and T. reesei GH3 β-Gs (78-94 kDa) in this study. Besides the molecular structure, the electric charge of enzyme might cause the changes in the free enzyme distribution and is further discussed in the following section.

Effect of pH on Enzyme Adsorption onto Lignin:

Although the hydrophobic interaction is known to be the primary driving force, the electrostatic interaction also affects the protein adsorption behavior onto the solid surface (Brash and Horbett, 1995). The contribution of electrostatic interactions between cellulase and lignin was studied by varying the pHs of the incubation buffer from pH 4.0 to 6.0. The results showed the unadsorbed protein of Ctec2 in the supernatant increased from 36.8 to 83.7% as the pH increased. Among the enzyme components, the change in the free activity was most pronounced for β-G at elevated pH. The β-G activity was not detectable at pH 4.0. However it was remarkably increased to 67.6% at pH 6.0. The highly pH-dependent β-G adsorption to lignin suggested the strong contribution of electrostatic interactions. As shown in Table 11, β-Gs from T. reesei have pI values of 5.7-6.4 and are positively charged at <pH 5.5. For lignin, the surface has been observed to be negatively charged (Dong et al., 1996; Lou et al., 2013; Nakagame et al., 2011; Rahikainen et al., 2013). When the zeta potentials of lignins from steam- or dilute acid pretreated softwoods were measured, the values were in the ranges from −21.5 to −13.5 mV at pH 4.8 (Lou et al., 2013; Nakagame et al., 2011). This explains that the attraction between the positively charged T. reesei β-G and the negatively charged lignins at low pH 4.0-5.0 results in the adsorption of most of β-G to lignin. Thus, when pH approaches the pI of β-G, the less positively charged protein results in the less adsorption onto lignins.

Compared to β-G, the increase in the free CBH and EG at higher pH was minimal probably due to the lower overall electrical charges. Major cellulases such as CBHI (pI 4.6), CBHII (pI 5.1), EGI (pI 4.7) and EGII (pI 4.9) have negative or neutral charges at pH 4.8-6.0. Also the CBM of cellulase interacting with lignin through hydrophobic interaction might result in the less sensitivity to pH changes. The effect of CBM on the adsorption of cellulase to lignin at different pHs was discussed by Rahikainen et al. (2013), and is consistent with adsorption patterns observed for lignin derived from LHW pretreated hardwoods.

Effect of NaCl on Enzyme Adsorption onto Lignin

The effect of salt ions on the enzyme adsorption onto lignin was studied by varying NaCl concentration from 0 to 400 mM at pH 4.8. The total free proteins in the supernatant increased from 50.7 to 81.3% in the presence of 200 mM NaCl. While EG activity was remained constant as 50-60%, there was a sudden increase of CBH activity at 200 mM NaCl by 19.7%. For β-G, its free activity gradually increased from 2.0 to 38.3%. The increase in free enzyme activities at higher NaCl concentration may reflect the decreased electrostatic effect on adsorption. The charged residues of protein and lignin become shielded by competing salt ions thereby resulting in the decreased electrostatic attraction. Above 200 mM of NaCl, ˜60% of β-G and ˜50% of EG still adsorbed to lignin. This shows that the hydrophobic interaction as a primary driving force still promotes enzyme adsorption to lignin when the electrostatic interaction was reduced. Similar to our result, Tu et al. (2009) found that cellulase adsorption onto steam exploded softwood lignin decreased as NaCl concentration increased.

Enhancement in Enzymatic Hydrolysis of Lignocellulose

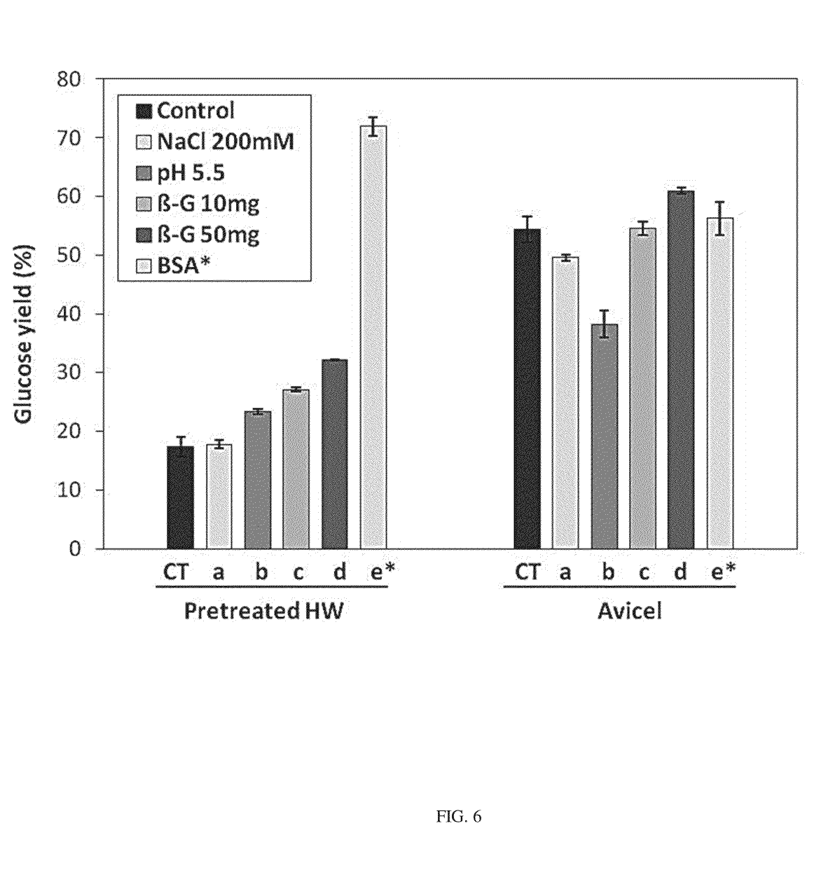

The enzyme interaction with hardwood lignin showed that T. reesei β-G was most strongly adsorbed and highly dependent on pH and ionic strength. In this work, the lignocellulosic substrate was enzymatically hydrolyzed in order to study how the non-productive adsorption affected the enzymatic hydrolysis efficiency. The glucose yield of LHW pretreated hardwood containing 57.9% glucan and 40.3% lignin was measured for a low enzyme loading amount of 5 FPU (8 mg) Ctec2/g glucan at given conditions (FIG. 6). While the enzymatic hydrolysis in the presence of 200 mM NaCl at pH 4.8 did not affect the glucose yield, the increase in pH from 4.8 to 5.5 improved the glucose yield by 6.0% (FIG. 6, points a-b). Although the non-productive adsorption of enzyme onto lignin was reduced at above conditions, the enzymatic hydrolysis efficiency was not significantly improved. This might be due to the decreased cellulase activity at pH 5.5 as shown in microcrystalline cellulose (Avicel) hydrolysis. The overall decrease in T. reesei cellulase cocktail (Ctec2) adsorption onto lignins at elevated pHs was in agreement with the results by Lou et al. (2013). The reduced nonproductive adsorption led to the enhanced glucose yield more than twofold when the dilute acid or sulfite pretreated softwood was hydrolyzed with higher amount (10 FPU/g glucan) of cellulase (Lou et al., 2013).

Since most of T. reesei β-G in Ctec2 was adsorbed onto lignin, Novozyme 188 (A. niger β-G) which was more resistant to soluble lignin inhibitors and adsorption onto insoluble lignins was added to compensate for the lost β-G activity. When supplemented with 10 and 50 mg protein/g glucan of A. niger β-G, the glucose yield increased by a factor of ˜1.8 (FIG. 6, points c-d). However the glucose yield could be increased up to 71.9% (by a factor of 4.1) when the hydrophobic lignin surface was blocked by BSA (FIG. 6, point e). It means that the non-productive adsorption of CBH and EG onto lignin coincides with reduced enzymatic hydrolysis at a relatively low enzyme loading of 8 mg protein/g glucan, which is equivalent to 4.7 mg protein/g-total pretreated solids.

The loss of cellulase activity due to the non-productive adsorption of cellulase enzymes onto lignin results in the dramatically reduced enzymatic hydrolysis efficiency of lignocellulose at enzyme loading of 8 mg protein/g glucan. Lignins derived from hardwoods pretreated at increasing severities of liquid hot water adsorbed enzymes more strongly. While the adsorption of enzyme was affected by the lignin structure, its degree and specificity were also highly dependent on pH and NaCl concentration. The non-productively adsorbed enzyme, especially T. reesei β-G, was reduced when pH was increased above 5.5 and conversely, addition of A. niger β-G increased hydrolysis by a factor of 2. These results show that adsorption of β-G onto lignin is a major factor that indirectly suppresses enzymatic hydrolysis of cellulose.

Those skilled in the art will recognize that numerous modifications can be made to the specific implementations described above. The implementations should not be limited to the particular limitations described. Other implementations may be possible.

### REFERENCES

1. Kim Y, Hendrickson R, Mosier N S, Ladisch M R (2009) Liquid hot water pretreatment of cellulosic biomass. Methods in Molecular Biology: Biofuels, ed Mielenz J R (The Humana Press, Totowa), 581: pp 93-102.
2. Lau M W, Dale B E (2009) Cellulosic ethanol production from AFEX-treated corn stover using Saccharomyces cerevisiae 424A (LNH-ST). Proc Natl Acad Sci USA 106:1368-1373.
3. Ladisch M R, Kohlmann K, Westgate P, Weil J, Yang Y (1998) Processes for treating cellulosic material. U.S. Pat. No. 5,846,787.
4. Söderström J, Galbe M, Zacchi G (2004) Effect of washing on yield in one- and two-step steam pretreatment of softwood for production of ethanol. Biotechnol Prog 20:744-749.
5. Nguyen Q A, Tucker M P, Keller F A, Eddy F P (2000) Two-stage dilute acid pretreatment of softwoods. Appl Biochem Biotechnol 84-86:561-576.
6. Kim Y, Kreke T, Mosier N S, Ladisch M R (2013) Severity factor coefficients for subcritical liquid hot water pretreatment of hardwood chips. Biotechnol Bioeng 111:254-263.
7. Overend R P, Chornet E, Gascoigne J A (1987) Fractionation of lignocelluloses by steam aqueous pretreatments. Philos Trans R Soc A 321:523-536.
8. Chang V S, Holtzapple M T (2000) Fundamental factors affecting biomass enzymatic reactivity. Appl Biochem Biotechnol 84-86:5-37.
9. Zhang Y H P, Lynd L (2004) Toward an aggregated understanding of enzymatic hydrolysis of cellulose: Non-complexed cellulase systems. Biotechnol Bioeng 88:797-824.
10. Zhu Z, et al. (2009) Comparative study of corn stover pretreated by dilute acid and cellulose solvent-based lignocellulose fractionation: Enzymatic hydrolysis, supramolecular structure, and substrate accessibility. Biotechnol Bioeng 103:715-24.
11. Kumar R, Wyman C E (2009) Access of cellulase to cellulose and lignin for poplar solids produced by leading pretreatment technologies. Biotechnol Prog 25:807-819.
12. Rollin J A, Zhu Z, Sathitsuksanoh N, Zhang Y H P (2011) Increasing cellulose accessibility is more important than removing lignin: A comparison of cellulose solvent-based lignocellulose fractionation and soaking in aqueous ammonia. Biotechnol Bioeng 108:22-30.
13. Del Rio L F, Chandra R P, Saddler J N (2011) The effects of increasing swelling and anionic charges on the enzymatic hydrolysis of organosolv-pretreated softwoods at low enzyme loadings. Biotechnol Bioeng 108:1549-1558.
14. Arantes V, Saddler J N (2011) Cellulose accessibility limits the effectiveness of minimum cellulase loading on the efficient hydrolysis of pretreated lignocellulosic substrates. Biotechnol Biofuels 4:3.
15. Jeoh T, et al. (2007) Cellulase digestibility of pretreated biomass is limited by cellulose accessibility. Biotechnol Bioeng 98: 112-122.
16. Puri V P (1984) Effect of crystallinity and degree of polymerization of cellulose on enzymatic saccharification. Biotechnol Bioeng 26:1219-1222.
17. Caulfield D F, Moore W E (1974) Effect of varying crystallinity of cellulose on enzymatic hydrolysis. Wood Sci 6:375-379.
18. Kumar R, Mago G, Balan V, Wyman C E (2009) Physical and chemical characterizations of corn stover and polar solids resulting from leading pretreatment technologies. Bioresour Technol 100:3948-3962.
19. Ladisch M R, Ximenes E, Kim Y, Mosier N S (2013) Biomass Chemistry. Catalysis for the conversion of biomass and its derivatives, eds Behresn M, Datye A K (Edition Open Access, Berlin), pp 131-164.
20. Ximenes E, Kim Y, Ladisch M R (2013) Biological Conversion of Plants to Fuels and Chemicals and the Effects of Inhibitors. Aqueous Pretreatment of Plant Biomass for Biological and Chemical Conversion to Fuels and Chemicals, ed Wyman C E (Wiley, New York), pp 39-60.
21. Zeng M, et al. (2011) Tissue-specific biomass recalcitrance in corn stover pretreated with liquid hot-water: Enzymatic Hydrolysis, Part 1. Biotechnol Bioeng 109: 390-397.
22. Zeng M, et al. (2011) Tissue-specific biomass recalcitrance in corn stover pretreated with liquid hot-water: Enzymatic Hydrolysis, Part 2. Biotechnol Bioeng 109: 398-404.
23. Zeng M, Mosier N S, Huang C-P, Sherman D M, Ladisch M R (2007) Microscopic examination of changes of plant cell structure in corn stover due to cellulase activity and hot water pretreatment. Biotechnol Bioeng 97: 265-278.
24. Chandra R P, Esteghlalian A R, Saddler J N (2008) Assessing substrate accessibility to enzymatic hydrolysis by cellulases. Characterization of lignocellulosic materials, ed Hu T Q (Blackwell, Oxford), pp 60-80.
25. Wong K K Y, Deverell K F, Mackie K L, Clark T A, Donaldson L A (1988) The relationship between fiber porosity and cellulose digestibility in steam exploded Pinus radiata. Biotechnol Bioeng 31:447-456.
26. Grethlein H E (1985) The effect of pore size distribution on the rate of enzymatic hydrolysis of cellulosic substrates. Nat Biotechnol 3:155-160.
27. Burns D S, Ooshima H, Converse A O (1989) Surface area of pretreated lignocellulosics as a function of the extent of enzymatic hydrolysis. Appl Biochem Biotechnol 20-21:79-94.
28. Ishizawa C, Davis M F, Schell D F, Johnson D K (2007) Porosity and its effect on the digestibility of dilute sulfuric acid pretreated corn stover. J Agric Food Chem 55:2575-2581.
29. Mansfield S D, Mooney C, Saddler J N (1999) Substrate and enzyme characteristics that limit cellulose hydrolysis. Biotechnol Prog 15:804-816.
30. TAPPI (1991) TAPPI useful method UM256. Water retention value (WRV). TAPPI Useful Methods, (TAPPI Press, Atlanta).
31. Chandra R P, et al. (2009). Comparison of methods to assess the enzyme accessibility and hydrolysis of pretreated lignocellulosic substrates. Biotechnol Lett 31:1217-1222.
32. Pan X, Xie D, Gilkes N, Gregg D J, Saddler J N (2005) Strategies to enhance the enzymatic hydrolysis of pretreated softwood with high residual lignin content. Appl Biochem Biotechnol 124:1069-1079.
33. Yang B, Wyman C E (2006) BSA treatment to enhance enzymatic hydrolysis of cellulose in lignin containing substrates. Biotechnol Bioeng 5:611-617.
34. Vidal Jr. B C, Dien B S, Ting K C, Singh V (2011) Influence of feedstock particle size on lignocellulose conversion. Appl Biochem Biotechnol 164:1405-1421.
35. Battista A, Coppicio S, Howsmon J A, Morehead F F, Sisson W A (1956) Level-off degree of polymerization relation to polyphase structure of cellulose fibers. Ind Eng Chem 48:333-335.
36. Fan L T, Lee Y-H, Beardmore D H (1980) Mechanism of the enzymatic hydrolysis of cellulose: effects of major structural features of cellulose on enzymatic hydrolysis. Biotechnol Bioeng 22:177-199.
37. Walseth C S (1952) The influence of the fine structure of cellulose on the action of cellulases. Tappi J 35:233-236.
38. Lee Y H, Fan L T (1983) Kinetic studies of enzymatic hydrolysis of insoluble cellulose: (II). Analysis of extended hydrolysis times. Biotechnol Bioeng 25:939-966.
39. Ladisch M R, et al. (1992) Intercalation in the pretreatment of cellulose in harnessing biotechnology for the 21^{st} century. Proceedings of the 9^{th} international biotechnology symposium and exposition, eds Ladisch M R, Bose A (American Chemical Society, Washington D.C.), pp 510-518.
40. Kim Y, Ximenes E, Mosier N S, Ladisch M R (2011) Soluble inhibitors/deactivators of cellulase enzymes from lignocellulosic biomass. Enzyme Microb Tech 48:408-415.
41. Ximenes E, Kim Y, Mosier N S, Dien B, Ladisch M R (2011) Deactivation of cellulases by phenols. Enzyme Microb Tech 48:54-60.
42. Ximenes E, Kim Y, Mosier N S, Dien B, Ladisch M R (2010) Inhibition of cellulase by phenols. Enzyme Microb Tech 46:170-176.
43. Kim Y, Kreke T, Hendrickson R, Parenti J, Ladisch M R (2013) Fractionation of cellulase and fermentation inhibitors from steam pretreated mixed hardwood. Bioresour Technol 135: 30-38.
44. Kwiatkowski J R, McAloon A J, Taylor F, Johnston D B (2006) Modeling of the process and costs of fuel ethanol production by the corn dry-grind process. Ind Crop Prod 23:288-296.
45. Gao D, et al. (2013) Increased enzyme binding to substrate is not necessary for more efficient cellulose hydrolysis. Proc Natl Acad Sci USA, early edition, 1213426110.
46. Kumar R, Wyman C E (2009) Cellulase adsorption and relationship to features of corn stover solids produced by leading pretreatments. Biotechnol Bioeng 103: 252-267.
47. Kumar R, Wyman C E (2009) Effects of cellulase and xylanase enzymes on the deconstruction of solids from pretreatment of poplar by leading technologies. Biotechnol Prog 25: 302-314.
48. Hames B D (1981) Gel electrophoresis of proteins: a practical approach. Eds Hames B D, Rickwood D (IRL Press, Washington D.C.) pp 1-92.
49. Ehrman T (1994) Standard method for the determination of extractives in biomass. Chemical analysis and testing task laboratory analytical procedures. NREL Ethanol Project.
50. Ehrman T (1994) Standard method for ash in biomass. Chemical analysis and testing task laboratory analytical procedures. NREL Ethanol Project.
51. Sluiter A, et al. (2006) Determination of structural carbohydrates and lignin in biomass. Biomass analysis technology team laboratory analytical procedures. NREL Biomass Program.
52. Sluiter A, et al. (2005) Determination of sugars, byproducts, and degradation products in liquid fraction process samples. Biomass analysis technology team laboratory analytical procedures (LAP 014). NREL Biomass Program.
53. Hong J, Ladisch M R, Gong C S, Wankat P C, Tsao G T (1981) Combined product and substrate inhibition equation for cellobiase. Biotechnol Bioeng 23:2779-2788.
54. Gong C S, Ladisch M R, Tsao G T (1977) Cellobiase from Trichoderma viside: purification, properties, kinetics and mechanism. Biotechnol Bioeng 19:959-981.
55. Kim Y, Mosier N S, Ladisch M R (2009) Enzymatic digestion of liquid hot water pretreated hybrid poplar. Biotechnol Prog 25:340-348.
56. Chandra R, Ewanick S, Hsieh C, Saddler J N (2008) The characterization of pretreated lignocellulosic substrates prior to enzymatic hydrolysis, part 1: A modified Simons' staining technique. Biotechnol Prog 24:1178-1185.
57. Yu X, Atalla R H (1998) A staining technique for evaluating the pore structure variations of microcrystalline cellulose powders. Power Technol 98:135-138.
58. Hubbell C A, Ragauskas A J (2010) Effect of acid-chlorite delignification on cellulose degree of polymerization. Bioresour Technol 101:7410-7415.
59. ASTM (1986) Standard test methods for intrinsic viscosity of cellulose (D 1795). American Society for Testing Materials, pp 360-366.
60. Heiningen A V, Tunc M S, Gao Y, Perez D D S (2004) Relationship between alkaline pulp yield and the mass fraction and degree of polymerization of cellulose in pulp. J Pulp Pap Sci 30:211-217.
61. Bonawitz N D, Kim J I, Tobimatsu Y T, Ciesielski P N, Anderson N A, Ximenes E, Maeda J, Ralph J, Donohoe B S, Ladisch M, Chapple C. 2014. Disruption of mediator rescues the stunted growth of a lignin-deficient Arabidopsis mutant. Nature (In press).
62. Carvalheiro F, Silva-Fernandes T, Duarte L C, Gírio F M. 2009. Wheat straw autohydrolysis: Process optimization and products characterization. Appl Biochem Biotechnol 153:84-93.
63. Chapple C, Ladisch M, Meilan R. 2007. Loosening lignin's grip on biofuel production. Nat Biotechnol 25:746-748.
64. Chen F, Dixon R A. 2007. Lignin modification improves fermentable sugar yields for biofuel production. Nat Biotechnol 25:759-761.
65. Chua M G S, Wayman M. 1979a. Characterization of autohydrolysis aspen (P. tremuloides) lignins. Part 1. Composition and molecular weight distribution of extracted autohydrolysis lignin. Can J Chem 57:1141-1149.
66. Chua M G S, Wayman M. 1979b. Characterization of autohydrolysis aspen (P. tremuloides) lignins. Part 3. Infrared and ultraviolet studies of extracted autohydrolysis lignin. Can J Chem 57:2603-2611.
67. Donohoe B S, Decker S R, Tucker M P, Himmel M E, Vinzant T B. 2008. Visualizing lignin coalescence and migration through maize cell walls following thermochemical pretreatment. Biotechnol Bioeng 101:913-925.
68. Eriksson T, Börjesson J, Tjerneld F. 2002. Mechanism of surfactant effect in enzymatic hydrolysis of lignocellulose. Enzyme Microb Technol 31:353-364.
69. Funaoka M, Shibata M, Abe I. 1990. Structure and depolymerization of acid-condensed lignin. Holzforschung 44:357-366.
70. Gellerstedt G, Henriksson G. 2008. Lignins: major sources, structure and properties. In: Belgacem M, Gandini A, editors. Monomers, polymers and composites from renewable resources. Amsterdam: Elsevier. p 201-224.
71. Ghose T K. 1987. Measurement of cellulase activities. Pure & Appl Chem 59:257-268.
72. Glasser W G, Barnett C A, Muller P C, Sarkanen, K V. 1983. The chemistry of several novel bioconversion lignins. J Agri Food Chem 31:921-930.
73. Glasser W G. 2000. Classification of lignin according to chemical and molecular structure. In: Glasser W G, Northey R A, Shultz T P, editors. Lignin: historical, biological, and materials perspectives. Washington D.C.: American chemical society. p 216-238.
74. Gupta R, Lee Y Y. 2010. Investigation of biomass degradation mechanism in pretreatment of switchgrass by aqueous ammonia and sodium hydroxide. Bioresour Technol 101:8185-8191.
75. Hansen M A T, Kristensen J B, Felby C, Jørgensen H. 2011. Pretreatment and enzymatic hydrolysis of wheat straw (Triticum aestivuum L.)—the impact of lignin relocation and plant tissues on enzymatic accessibility. Bioresour Technol 102:2804-2811.
76. Hatakeyama T, Hatakeyama H. 2004. Thermal properties of green polymers and biocomposites. Dortrecht: Kluwer academic publishers. p 171-215.
77. Hatakayama H, Tsujimoto Y, Zarubin M J, Krutov S M, Hatakeyama T. 2010. Thermal decomposition and glass transition of industrial hydrolysis lignin. J Therm Anal Calorim 101:289-295.
78. Hergert H L. 1971. Infrared spectra. In: Sarkanen K V, Ludwig C H, editors. Lignins: occurrence, formation, structure and reactions. New York: Wiley-Interscience. p 267-293.
79. Hu F, Jung S, Ragauskas A. 2012. Pseudo-lignin formation and its impact on enzymatic hydrolysis. Bioresour Technol 117:7-12.
80. Irvine G M. 1985. The significance of the glass transition of lignin in thermomechanical pulping. Wood Sci Technol 19:139-149.
81. Jakobsons J, Hording B, Erins P, Sundquist J. 1995. Characterization of alkali soluble fraction of steam exploded birch wood. Holzforschung 49:51-59.
82. Kelly S S, Rials T G, Glasser W G. 1987. Relaxation behavior of the amorphous components of wood. J Materials Sci 22:617-624.
83. Kim Y, Kreke T, Mosier N S, Ladisch M R. 2013. Severity factor coefficients for subcritical liquid hot water pretreatment of hardwood chips. Biotechnol Bioeng 9999:1-10.
84. Kishimoto T, Chiba W, Saito K, Fukushima K, Uraki Y, Ubukata M. 2010. Influence of syringyl to guaiacyl ratio on the structure of natural and synthetic lignins. J Agri Food Chem. 58:895-901.
85. Ko J K, Ximenes E, Kim Y, Ladisch M R. 2014. Adsorption of enzyme onto lignins of liquid hot water pretreated hardwoods. Biotechnol Bioeng (Submitted)
86. Kristensen J B, Borjesson J, Bruun M H, Tjerneld F, Jørgensen H. 2007. Use of surface active additives in enzymatic hydrolysis of wheat straw lignocellulose. Enzyme Microb Technol 40:888-895.
87. Kubo S, Kadla J F. 2005. Hydrogen bonding in lignin: A fourier transform infrared model compound study. Biomacromolecules 6:2815-2821.
88. Kumar L, Arantes V, Chandra R, Saddler J. 2012. The lignin present in steam pretreated softwood binds enzymes and limits cellulose accessibility. Bioresour Technol 103:201-208.
89. Kumar R, Hu F, Sannigrahi P, Jung S, Ragauskas A J, Wyman C E. 2013. Carbohydrate derived-pseudo-lignin can retard cellulose biological conversion. Biotechnol Bioeng 110:737-753.
90. Li J, Henriksson G, Gellerstedt G. 2005. Carbohydrate reactions during high-temperature steam treatment of aspen wood. Appl Biochem Biotechnol. 125:175-188.
91. Li J, Henriksson G, Gellerstedt G. 2007. Lignin depolymerization/repolymerizaiton and its critical role for delignification of aspen wood by steam explosion. Bioresour Technol 98:3061-3068.
92. Lindeberg 0, Walding J. 1987. Reactions of nitrated kraft lignin in an alkaline oxygen bleaching stage. Tappi J 70:119-123.
93. Nada A M A, Yousef M A, Shaffei K A, Salah A M. 1998. Infrared spectroscopy of some treated lignins. Polym Degrad Stabil 62:157-163.
94. Nakagame S, Chandra R P, Saddler J N. 2010. The effect of isolated lignins, obtained from a range of pretreated lignocellulosic substrates, on enzymatic hydrolysis. Biotechnol Bioeng 105:871-879.
95. Nakagame S, Chandra R P, Saddler J N. 2011a. The influence of lignin on the enzymatic hydrolysis of pretreated biomass substrates. In: Zhu J Y, Zhang X, Pan X J, editor. Sustainable production of fuels, chemicals, and fibers from forest biomass. Washington D.C.: American Chemical Society. p 145-167.
96. Nakagame S, Chandra R P, Kadla J F, Saddler J N. 2011b. The isolation, characterization and effect of lignin isolated from steam pretreated Douglas-fir on the enzymatic hydrolysis of cellulose. Bioresour Technol 102:4507-4517.
97. Obst J R. 1982. Guaiacyl and syringyl lignin composition in hardwood cell components.

Holzforschung 36:143-152.
98. Olsson A-M, Salmén L. 1992. Viscoelasticity of biomaterials. ACS Symp Ser 489:133-143.
99. Pan X-J, Sano Y. 2000. Comparison of acetic acid lignin with milled wood and alkaline lignins from wheat straw. Holzforschung 54:61-65.
100. Pandey K K. 1998. A study of chemical structure of soft and hardwood and wood polymers by FTIR spectroscopy. J Appl Polym Sci 71:1969-1975.
101. Pingali S V, Urban V S, Heller W T, McGaughey J, O'Neill H, Foston M, Myles D A, Ragausks A, Evans B R. 2010. Breakdown of cell wall nanostructure in dilute acid pretreated biomass. Biomacromolecules 11:2329-2335.
102. Saito T, Brown R H, Hunt M A, Pickel D L, Pickel J M, Messman J M, Baker F S, Keller M, Naskar A K. 2012. Turning renewable resources into value-added polymer: development of lignin-based thermoplastic. Green Chem 14:3295-3303.
103. Samuel R, Pu Y, Raman B, Ragauskas A J. 2010. Structural characterization and comparison of switchgrass ball-milled lignin before and after dilute acid pretreatment. Appl Biochem Biotechnol 162:62-74.
104. Sannigrahi P, Ragaukus A J, Miller S J. 2008. Effects of two-stage dilute acid pretreatment on the structure and composition of lignin and cellulose in loblolly pine. Bioenerg Res 1:205-214.
105. Schmidl G W. 1992. Ph.D. Thesis: Molecular weight characterization and rheology of lignin for carbon fibers. Gainesville, Fla., USA: University of Florida.
106. Selig M J, Viamajala S, Decker S R, Tucker M P, Himmel M E, Vinzant T B. 2007. Deposition of lignin droplets produced during dilute acid pretreatment of maize stems retards enzymatic hydrolysis of cellulose. Biotechnol Prog 23:1333-1339.
107. Sipos B, Dienes D, Schleicher A, Perazzini R, Crestini C, Siika-aho M, Réczey K. 2010. Hydrolysis efficiency and enzyme adsorption on steam-pretreated spruce in the presence of poly(ethylene glycol). Enzyme Microb Technol 47:84-90.
108. Sjöström E. 1981. Wood chemistry: fundamentals and applications. New York: Academic press, Inc. p 71-88.
109. Sluiter A, Hames B, Ruiz R, Scarlata C, Sluiter J, Templeton D, Crocker D. 2008. Determination of structural carbohydrates and lignin in biomass. Golden, Colo.: National Renewable Energy Laboratory.
110. Stewert J J, Akiyama T, Chapple C, Ralph J, Mansfield S D. 2009. The effects on lignin structure of overexpression of ferulate 5-hydroxylase in hybrid poplar. Plant Physiol 150:621-635.
111. Tu M, Chandra R P, Saddler J N. 2007. Recycling cellulases during the hydrolysis of steam exploded and ethanol pretreated lodgepole pine. Biotechnol Prog 23:1130-1137.
112. Yasuda S, Ota K. 1987. Chemical structures of sulfuric acid lignin. X. Reaction of syringylglycerol-β-syringyl ether and condensation of syringyl nucleus with guaiacyl lignin model compounds in sulfuric acid. Holzforschung 41:59-65.
113. Yasuda S, Hirano J. 1990. Chemical structures of sulfuric acid lignin. XI. Physical and chemical properties of beech sulfuric acid lignin. Mokuzai Gakkaishi 36:454-459.
114. Yasuda S, Fukushima K, Kakehi A. 2001. Formation and chemical structures of acid-soluble lignin I: sulfuric acid treatment time and acid-soluble lignin content of hardwood. J Wood Sci 47:69-72.
115. Yoshida H, Mörck R, Kringstad K P. 1987. Fractionation of kraft lignin by successive extraction with organic solvents. II. Thermal properties of kraft lignin fractions. Holzforschung 41:171-176.
116. Zhu J Y, Pan X J. 2010. Woody biomass pretreatment for cellulosic ethanol production: Technology and energy consumption evaluation. Bioresour Technol 101: 4992-5002.
117. Berlin A, Balakshin M, Gilkes N, Kadla J, Maximenko V, Kubo S, Saddler J. 2006. Inhibition of cellulase, xylanase and β-glucosidase activities by softwood lignin preparations. J Biotechnol 125:198-209.
118. Börjesson J, Engqvist M, Sipos B, Tjerneld F. 2007. Effect of poly(ethylene glycol) on enzymatic hydrolysis and adsorption of cellulase enzymes to pretreated lignocellulose. Enzyme Microb Technol 41:186-195.
119. Brash J L, Horbett T A. 1995. Proteins at Interfaces II—an overview. In: Horbett T A, Brash J L, editors. Proteins at Interfaces II. ACS Symposium Series 602. Washington D.C.: p 1-23.
120. Chirico W J, Brown R D. 1987. Purification and characterization of a β-glucosidase from Trichoderma reesei. Eur J Biochem 165:333-341.
121. Deshpande M V, Eriksson K-E, Pettersson L G. 1984. An assay for selective determination of exo-1,4-β-glucanases in a mixture of cellulolytic enzymes. Anal Biochem 138:481-487.
122. Dong D, Fricke A L, Moudgil B M, Johnson H. 1996. Electrokinetic study of kraft lignin. Tappi J 79:191-197.
123. Foreman P K, Brown D, Dankmeyer L, Dean R, Diener S, Dunn-Coleman N S, Goedegebuur F, Houfek T D, England G J, Kelly A S, Meerman H J, Mitchell T, Mitchinson C, Olivares H A, Teunissen P J M, Yao J, Ward M. 2003. Transcriptional regulation of biomass-degrading enzymes in the filamentous fungus Trichoderma reesei. J Biol Chem 278:31988-31997.
124. Häkkinen M, Arvas M, Oja M, Aro N, Penttila M, Saloheimo M, Pakula™. 2012. Re-annotation of the CAZy genes of Trichoderma reesei and transcription in the presence of lignocellulosic substrates. Microb Cell Fact 11:134
125. Haven MØ, Jørgensen H. 2013. Adsorption of β-glucosidases in two commercial preparations onto pretreated biomass and lignin. Biotechnol Biofuels 6:165-179
126. Hui J P M, Lanthier P, White T C, McHugh S G, Yaguchi M, Roy R, Thibault P. 2001. Characterization of cellobiohydrolase I (Cel7A) glycoforms from extracts of Trichoderma reesei using capillary isoelectric focusing and electrospray mass spectrometry. J Chromatogr B 752:349-368.
127. Kemmer G, Keller S. 2010. Nonlinear least-squares data fitting in Excel spreadsheets. Nat Protoc 5:267-281.
128. Ko J K, Kim Y, Ximenes E, Ladisch M R. 2014. Effect of liquid hot water pretreatment severity on properties of hardwood lignin and enzymatic hydrolysis of cellulose. Biotechnol Bioeng (Submitted)
129. Kyriacou A, Neufeld R J, MacKenzie CR. 1988. Effect of physical parameters on the adsorption characteristics of fractionated Trichoderma reesei cellulase components. Enzyme Microb Technol 10:675-681.
130. Kyte J, Doolittle R F. 1982. A simple method for displaying the hydropathic character of a protein. J Mol Biol 157:105-132.
131. Lima M A, Oliveira-Neto M, Kadowaki M A S, Rosseto F R, Prates E T, Squina F M, Leme A F P, Skaf M S, Polikarpov I. 2013. Aspergillus niger β-glucosidase has a cellulase-like tadpole molecular shape: insights into GH3 β-glucosidases structure and function. J Biol Chem 288:32991-33005.
132. Lou H, Zhu J Y, Lan T Q, Lai H, Qiu X. 2013. pH-induced lignin surface modification to reduce nonspecific cellulase binding and enhance enzymatic saccharification of lignocelluloses. ChemSusChem 6:919-927.
133. Medve J, Lee D, Tjerneld F. 1998. Ion-exchange chromatographic purification and quantitative analysis of Trichoderma reesei cellulases cellobiohydrolase I, II and endoglucanase II by fast protein liquid chromatography. J Chromatogr A 808:153-165.
134. Medve J, Stålberg J, Tjerneld F. 1997. Isotherms for adsorption of cellobiohydrolase I and II from Trichoderma reesei on microcrystalline cellulose. Appl Biochem Biotech 66:39-56.
135. Nidetzky B, Steiner W, Claeyssens M. 1994. Cellulose hydrolysis by the cellulases from Trichoderma reesei: adsorptions of two cellobiohydrolases, two endocellulases and their core proteins on filter paper and their relation to hydrolysis. Biochem J 303:817-823.
136. Norde W. 1986. Adsorption of proteins from solution at the solid-liquid interface. Adv Colloid Interface Sci 25:267-340.
137. Norde W. 1996. Driving forces for protein adsorption at solid surfaces. Macromol Symp 103:5-18.
138. Ooshima H, Burns D S, Converse A O. 1990. Adsorption of cellulase from Trichoderma reesei on cellulose and lignacious residue in wood pretreated by dilute acid with explosive decompression. Biotechnol Bioeng 36:446-452.
139. Ouyang J, Yan M, Kong D, Xu L. 2006. A complete protein pattern of cellulase and hemicellulose genes in the filamentous fungus Trichoderma reesei. Biotechnol J 1:1266-1274.
140. Palonen H, Tjerneld F, Zacchi G, Tenkanen M. 2004. Adsorption of Trichoderma reesei CBH I and EG II and their catalytic domains on steam pretreated softwood and isolated lignin. J Biotechnol 107:65-72.
141. Pedersen M, Johansen K S, Meyer A S. 2011. Low temperature lignocellulose pretreatment: effects and interactions of pretreatment pH are critical for maximizing enzymatic monosaccharide yields from wheat straw. Biotechnol Biofuels 4:1-10.
142. Rahikainen J, Mikander S, Marjamaa K, Tamminen T, Lappas A, Viikari L, Kruus K. 2011. Inhibition of enzymatic hydrolysis by residual lignins from softwood—study of enzyme binding and inactivation on lignin-rich surface. Biotechnol Bioeng 108:2823-2834.
143. Rahikainen J L, Evans J D, Mikander S, Kalliola A, Puranen T, Tamminen T, Marjamaa K, Kruus K. 2013. Cellulase-lignin interactions—The role of carbohydrate-binding module and pH in non-productive binding. Enzyme Microb Technol 53:315-321.
144. Sutcliffe R, Saddler J N. 1986. The role of lignin in the adsorption of cellulases during enzymatic treatment of lignocellulosic material. Biotechnol Bioeng Symp 17:749-762.
145. Tu M, Pan X, Saddler, J N. 2009. Adsorption of cellulase on cellulolytic enzyme lignin from lodgepole pine. J Agri Food Chem 57:7771-7778.
146. Vinzant T B, Adney W S, Decker S R, Baker J O, Kinter M T, Sherman N E, Fox J W, Himmel M E. 2001. Fingerprinting Trichoderma reesei hydrolases in a commercial cellulase preparation. Appl Biochem Biotechnol 91-93:99-107.

1. A method for enhancing enzyme hydrolysis, comprising:
pretreating a starting material at a temperature range to produce a pretreated starting material; and
introducing a non-catalytic protein into the pretreated starting material to thereby enhance e enzyme hydrolysis.

2. The method of claim 1, the enzyme is a cellulase enzyme.

3. The method of claim 1, the starting material is a cellulose-containing material.

4. The method of claim 1, the temperature range is about 220° C. to about 230° C.

5. The method of claim 1, the non-catalytic protein is derived from at least one of vegetable matter, food processing waste or co-product streams, corn (gluten and zein), corn processing effluent from dry mill, wet mill, or fermentation processes.

6. The method of claim 1, the vegetable matter comprises soybeans.

7. The method of claim 5, the fermentation processes utilize corn as a feedstock, residual protein form microbial fermentation of sugar cane or sugar beet to ethanol.
